(19) United States
(12) Patent Application Publication
(10) Pub. No.: US 2012/0270233 A1
(43) Pub. Date: Oct. 25, 2012

(54) ASSOCIATION OF BIOMARKERS WITH PATIENT OUTCOME

(75) Inventors: Donald E. Waldron, Fairfield, CT (US); Alpana Waldron, Fairfield, CT (US); Robert Pinard, Andover, MA (US); Mark Gustavson, Niantic, CT (US)

(73) Assignee: HistoRx, Inc.

(21) Appl. No.: 12/866,836

(22) PCT Filed: Feb. 10, 2009
(86) PCT No.: PCT/US2009/033691

§ 371 Date: Jun. 23, 2011

Related U.S. Application Data
(60) Provisional application No. 61/027,759, filed on Feb. 11, 2008.
(60) Provisional application No. 61/064,230, filed on Feb. 22, 2008.
(60) Provisional application No. 61/071,185, filed on Apr. 16, 2008.

(51) Int. Cl. G01N 21/64; C12Q 1/02; C12Q 1/48; G01N 21/17

(52) U.S. Cl. 435/7.4; 435/29; 435/15

(57) ABSTRACT

The present method relates to quantification of prognostic and predictive biomarkers of the PDK/AKT/mTOR pathway, such as GSK3β, S6, CREB, PTEN, AKT and mTOR, using AQUA® analysis to estimate both patient risk and benefit of treatment to patients diagnosed with glioblastoma. Unlike traditional IHC, the AQUA® system is objective and produces quantitative in situ protein expression data on a continuous scale. Taking advantage of the power of the AQUA system, the present method provides a highly robust and standardized diagnostic assays that can be used in the clinical setting to provide physicians with reliable prognostic and predictive information. Glioblastoma multiform (GBM) remains one of the most aggressive human cancers, and biomarkers that provide prognostic and predictive information would be extremely valuable to both the physician and the patient. A patient's risk may be determined using the prognostic biomarkers of the present method. Such a prognostic determination will allow physicians to identify patients with a relatively ‘good’ or a relatively ‘poor’ prognosis. The benefit of treating specific patients with a specific therapy, may be determined usin̂ the predictive markers of the present method. Treatment with the AGC-family kinase inhibitor enzastaurin, for example, identifies patients that will likely benefit from treatment or not.

### CROSS-REFERENCE TO RELATED PATENT APPLICATIONS

This application claims priority from U.S. provisional application No. 61/027,759, filed Feb. 11, 2008; U.S. provisional application No. 61/064,230 filed Feb. 22, 2008; and U.S. provisional application No. 61/071,185 filed Apr. 16, 2008, the disclosures of which are incorporated herein by reference in their entirety.

### BACKGROUND

Unlike traditional IHC, the AQUA® system is objective and produces strictly quantitative in situ protein expression data on a continuous scale. The AQUA® system takes advantage of the multiplexing power of fluorescence by using multiple markers to molecularly differentiate cellular and sub-cellular compartments within which simultaneous quantification of biomarkers-of-interest can be performed. In addition, AQUA analysis provides for standardization and a high degree of reproducibility with % CVs less than 5%, which is superior to any chromagen-based IHC quantification system available to date. Taking advantage of the power of the AQUA system, we wish to develop highly robust and standardized diagnostic assays that can be used in the clinical setting to provide physicians with reliable diagnostic information.

Glioblastoma multiforme (GBM) remains one of the most aggressive human cancers with median survival times of only 12-15 months. Biomarkers that provide prognostic information would be extremely valuable to both the physician and the patient. PTEN and to a lesser extent mTOR have been shown to have some prognostic value in predicting survival. To date, PTEN expression by categorical expression analysis (traditional immunohistochemistry (IHC)) and RT-PCR has been shown to correlate with better survival in glioblastoma (Sano, T et al. Differential Expression of MMAC/PTEN in Glioblastoma Multiforme: Relationship to Localization and Prognosis, 1999, CANCER RESEARCH 59, 1820-1824), a particularly aggressive form of brain cancer with median survival times of less than 15 months. Although, mTOR (a component of the PTEN pathway) in its phosphorylated active form has been shown to predict survival in GBM, total mTOR expression and its association with GBM survival has not been examined.

This assay is useful in segregating patient populations for treatment in both a predictive and prognostic manner. For example: Enzastaurin (LY317615.HCl) is a novel acyclic bisindolylmaleimide currently in phase 2 clinical trials in combination with temozolomide and radiation for the front-line treatment of glioblastoma multiforme. Enzastaurin is an ATP-competitive inhibitor of PKCI3, as well as, an inhibitor of other AGC-family kinases, including other PKC isoforms, p90RSK, GSK3β and p70S6K. In a wide array of human cancer cell lines, including glioblastoma cell lines, Enzastaurin treatment blocks signaling through the PI3 kinase/AKT/mTOR pathway. Accordingly, Enzastaurin suppresses the phosphorylation of GSK3Bser9, AKTser473, CREBser133 and the S6 ribosomal protein at ser235/236 and ser240/244. Additionally, rapamycin also functions to modulate the PI3 kinase/AKT/mTOR pathway by inhibiting mTOR.

### SUMMARY

The presently claimed method is applicable to identifying both prognostic and predictive biomarkers within the PI3K/AKT/mTOR signaling pathway. Prognostic biomarkers evaluate a patient's risk associated with a particular disease, regardless of therapy. Prognostic biomarkers identify patients that have either a statistically “good” or a “poor” prognosis. Predictive biomarkers evaluate the benefit of a specific treatment to patients. Clinically, predictive biomarkers allow selection of patients most likely to benefit from a specific treatment, while sparing patients whom would not benefit from suffering the toxic effects often associated with therapy. The present method can identify both prognostic biomarkers associated with disease risk and predictive biomarkers associated with treatment benefit.

As stated, prognostic biomarkers of the PI3k/AKT/mTOR pathway may be used to evaluate a patient's risk associated with a particular disease, regardless of therapy. More preferably, the prognostic biomarkers GSK3β, S6, CREB, PTEN, AKT, mTOR and pmTOR are used to identify patients identify patients that have either a statistically “good” or a “poor” prognosis.

In one embodiment, there is provided a method of determining a prognosis of a patient suffering from a medical condition comprising: an expression level of at least one protein biomarker, and/or a phosphorylated form thereof, associated with a PI3K/AKT/mTOR pathway in a tissue specimen obtained from the patient, and assessing the patient's prognosis from the determined expression level.

In one such embodiment, a method is described which comprises quantitatively assessing the concentration of protein biomarkers, and/or phosphorylated forms thereof, of the PI3k/AKT/mTOR pathway in a tissue specimen obtained from the patient, wherein the concentration levels protein biomarkers, and/or phosphorylated forms thereof, indicates the patient has either a relatively good prognosis or a relatively poor prognosis.

In one such embodiment, a method is described which comprises quantitatively assessing the concentration of PTEN and mTOR and/or pmTOR and/or pAKT protein biomarker in a tissue specimen obtained from the patient, wherein high levels of PTEN indicates the patient has a relatively good prognosis and wherein low levels of PTEN indicates the patient has a relatively poor prognosis.

In another embodiment, the method comprises quantitatively assessing the concentration of pAKT and PTEN and/or mTOR and/or pmTOR protein biomarker in a tissue specimen obtained from the patient, wherein high levels of pAKT indicates the patient has a relatively poor prognosis and wherein low levels of pAKT indicates the patient has a relatively good prognosis.

In one embodiment, there is provided a method of determining the prognosis of a patient. The method comprises quantitatively assessing the concentration of PTEN and mTOR protein biomarkers in a tissue specimen obtained from the patient, wherein high PTEN and high mTOR protein expression levels indicates the patient has a relatively good prognosis and wherein low PTEN and low mTOR, high PTEN and low mTOR, low PTEN and high mTOR levels of protein expression indicates the patient has a relatively poor prognosis.

In another embodiment, there is provided a method of determining the prognosis of a patient. The method comprises quantitatively assessing the concentration of PTEN and pAKT protein biomarkers in a tissue specimen obtained from the patient, wherein high AKT and low PTEN protein expression levels indicates the patient has a relatively very poor prognosis compared to low PTEN, low pAKT; low PTEN, medium pAKT; high PTEN, low pAKT; high PTEN, medium pAKT; and high PTEN, high pAKT protein expression levels.

In yet another embodiment there is provided a method of determining the prognosis or relative risk of a patient, the method comprises quantitatively assessing the concentration of PTEN, pAKT, mTOR, and pmTOR, protein biomarkers in a tissue specimen obtained from the patient, wherein expression or AQUA® score of each biomarker on a continuous scale is put into a Cox regression model for continuous variables resulting in a calculation of overall patient risk.

In yet another embodiment there is provided a method of determining the prognosis or relative risk of a patient, the method comprises quantitatively assessing the concentration of PTEN, pAKT, mTOR, and pmTOR, protein biomarkers in a tissue specimen obtained from the patient, wherein expression or AQUA® score of each biomarker is first categorized into low and high based on optimal univariate cutpoints, then applied to a Cox regression model for categorical variables resulting in a calculation of overall patient risk.

In one embodiment, there is provided a method of determining the prognosis of a patient. In one such embodiment, a method is described which comprises quantitatively assessing the concentration of the protein biomarkers GSK3B, S6, or CREB, and/or phosphorylated forms thereof, in a tissue specimen obtained from the patient, wherein high levels of phosphorylated GSK3B indicates the patient has a relatively poor prognosis and wherein low levels of phosphorylated GSK3B indicates the patient has a relatively good prognosis.

In one embodiment, there is provided a method of determining the prognosis of a patient. In one such embodiment, a method is described which comprises quantitatively assessing the concentration of the phosphorylated protein biomarkers GSK3B, S6, or CREB in a tissue specimen obtained from the patient, wherein high levels of phosphorylated S6 indicates the patient has a relatively poor prognosis and wherein low levels of phosphorylated S6 indicates the patient has a relatively good prognosis.

In one embodiment, there is provided a method of determining the prognosis of a patient. In one such embodiment, a method is described which comprises quantitatively assessing the concentration of the phosphorylated protein biomarkers GSK3B, S6, or CREB in a tissue specimen obtained from the patient, wherein high levels of phosphorylated CREB indicates the patient has a relatively poor prognosis and wherein low levels of phosphorylated CREB indicates the patient has a relatively good prognosis.

In one embodiment, there is provided a method of determining the prognosis of a patient. The method comprises quantitatively assessing the concentration of phosphorylated GSK3B, S6, or CREB protein biomarkers in a tissue specimen obtained from the patient, wherein phosphorylated GSK3B, S6, or CREB-high protein expression levels indicates the patient has a relatively poor prognosis and wherein phosphorylated GSK3B, S6, or CREB-low protein expression levels indicates the patient has a relatively good prognosis.

In yet another embodiment there is provided a method of determining the prognosis or relative risk of a patient, the method comprises quantitatively assessing the concentration of phosphorylated GSK3B, S6, or CREB, protein biomarkers in a tissue specimen obtained from the patient, wherein expression or AQUA® score of each biomarker on a continuous scale is put into a Cox regression model for continuous variables resulting in a calculation of overall patient risk.

In yet another embodiment there is provided a method of determining the prognosis or relative risk of a patient, the method comprises quantitatively assessing the concentration of phosphorylated GSK3B, S6, or CREB, protein biomarkers in a tissue specimen obtained from the patient, wherein expression or AQUA® score of each biomarker is first categorized into low and high based on optimal univariate cutpoints, then applied to a Cox regression model for categorical variables resulting in a calculation of overall patient risk.

In one embodiment, there is provided a method of determining the prognosis of a patient by quantitatively assessing the concentration of one or more biomarkers in a tissue sample. The method comprises: a) incubating the tissue sample with a first stain that specifically labels a first marker defined subcellular compartment, a second stain that specifically labels a second marker defined subcellular compartment and a third stain that specifically labels the biomarker; b) obtaining a high resolution image of each of the first, the second and the third stain in the tissue sample; c) assigning a pixel of the image to a first compartment based on the first stain intensity; a second compartment based on the second stain intensity; or to neither a first nor second compartment; d) measuring the intensity of the third stain in each of the pixels assigned to either the first or the second compartment or both; e) determining a staining score indicative of the concentration of the biomarker in the first or the second compartment or both; and f) plotting the biomarker concentration in relationship to a second biomarker concentration indicates the patient's prognosis.

In one embodiment, the biomarker is PTEN and a second biomarker is mTOR, wherein high expression of PTEN together with high expression of mTOR in a tissue sample is indicative of relatively good prognosis.

In another embodiment, the biomarker is PTEN and a second biomarker is pAKT, wherein low expression of PTEN together with high expression of pAKT in a tissue sample is indicative of relatively very poor prognosis.

A kit comprising one or more stains, each labeling a specific biomarker selected from the group consisting of: GSK3β, phosphorylated GSK2β, S6, phosphorylated S6, CREB, phosphorylated CREB, PTEN, AKT, phosphorylated pAKT, mTOR, phosphorylated mTOR optionally, a first stain specific for a first subcellular compartment of a cell, optionally, a second stain specific for a second subcellular compartment of the cell; and instructions for using the kit.

In one embodiment, there is provided a kit which comprises: a) a first stain specific for PTEN; b) a second stain specific for a first subcellular compartment of a cell; and c) instructions for using the kit.

In another embodiment, there is provided a kit which comprises: a) a first stain specific for mTOR; b) a second stain specific for a first subcellular compartment of a cell; and c) instructions for using the kit.

In one embodiment, there is provided a kit which comprises: a) a first stain specific for pAKT; b) a second stain specific for a first subcellular compartment of a cell; and c) instructions for using the kit.

In another embodiment, there is provided a kit which comprises: a) a first stain specific for pmTOR; b) a second stain specific for a first subcellular compartment of a cell; and c) instructions for using the kit.

In one embodiment, the biomarker is GSK3B and a second biomarker is specific for a first subcellular compartment of a cell, wherein high expression of GSK3B in a tissue sample is indicative of relatively poor prognosis.

In one embodiment, the biomarker is S6 and a second biomarker is specific for a first subcellular compartment of a cell, wherein high expression of S6 in a tissue sample is indicative of relatively poor prognosis.

In one embodiment, the biomarker is CREB and a second biomarker is specific for a first subcellular compartment of a cell, wherein high expression of CREB in a tissue sample is indicative of relatively poor prognosis.

In another embodiment, there is provided a kit which comprises: a) a first stain specific for GSK3B; b) a second stain specific for a first subcellular compartment of a cell; and c) instructions for using the kit.

In another embodiment, there is provided a kit which comprises: a) a first stain specific for S6; b) a second stain specific for a first subcellular compartment of a cell; and c) instructions for using the kit.

In another embodiment, there is provided a kit which comprises: a) a first stain specific for CREB; b) a second stain specific for a first subcellular compartment of a cell; and c) instructions for using the kit.

In one embodiment, there is provided a kit which comprises: a) a first stain specific for GSK3B; b) a second stain specific for a first subcellular compartment of a cell; and c) instructions for using the kit.

In one embodiment, there is provided a kit which comprises: a) a first stain specific for S6; b) a second stain specific for a first subcellular compartment of a cell; and c) instructions for using the kit.

In one embodiment, there is provided a kit which comprises: a) a first stain specific for CREB; b) a second stain specific for a first subcellular compartment of a cell; and c) instructions for using the kit.

In one embodiment, there is provided a method of identifying a patient suitable for treatment with a pharmaceutical inhibitor of the PI3k/AKT/mTOR pathway. Predictive biomarkers allow for separation of patients that may benefit from treatment with a pharmaceutical inhibitor of the PI3k/AKT/mTOR pathway from those that may not. The presently claimed method comprises a step of quantitatively assessing the concentration of one or more phosphorylated biomarkers in a tissue specimen obtained from the patient, wherein the levels of the one or more phosphorylated biomarkers indicates the patient is likely to benefit from treatment with the pharmaceutical inhibitor of the PI3k/AKT/mTOR pathway or not. In some embodiments of the method, the patient is naïve.

Predictive biomarkers may be used to identify patients suitable for treatment with a pharmaceutical inhibitor of the PI3k/AKT/mTOR pathway in any of the aforementioned embodiments, including both methods and kits, using prognostic biomarkers. Preferably, the predictive biomarkers GSK3β, S6, CREB, PTEN, AKT, mTOR and pmTOR are used to identify patients suitable for treatment with a pharmaceutical inhibitor of the PI3k/AKT/mTOR pathway. Preferably, the pharmaceutical inhibitor for treating a patient is selected from the group consisting of Rapamycin, Temsirolimus (Torisel), Everolimus (RAD001), AP23573, Bevacizumab, BIBW 2992, Cetuximab, Imatinib, Trastuzumab, Gefitinib, Ranibizumab, Pegaptanib, Sorafenib, Sasatinib, Sunitinib, Erlotinib, Nilotinib, Lapatinib, Panitumumab, Vandetinib, E7080, Sunitinib, Pazopanib, Enzastaurin, Cediranib, Alvocidib, Gemcitibine, Axitinib, Bosutinib, Lestartinib, Semaxanib, Vatalanib or combinations thereof. Preferably, the predictive biomarkers are selected from the group consisting of GSK3β, S6, CREB, PTEN, AKT and mTOR, and phosphorylated forms thereof, used to identify patients suitable for treatment with the aforementioned pharmaceutical inhibitors. Most preferably, the pharmaceutical inhibitors are Enzastaurin or rapamycin, optionally combined with temozolomide and radiation.

In on embodiment the expression level of at least one protein biomarker associated with a PI3K/AKT/mTOR pathway is characterized as low, medium or high.

In on embodiment the expression level of said biomarker is expressed as an AQUA® score by which said patient's expression level may be characterized as relatively low, intermediate or high based on unsupervised cluster analysis of AQUA® scores from a population of patients with said medical condition.

In on embodiment a low to intermediate AQUA® score for nuclear expression of GSK3β ranges from about 300 to about 2000.

In on embodiment a high AQUA® score for nuclear expression of GSK3β ranges from about 2000 to about 4000.

In on embodiment a low to intermediate AQUA® score for cytoplasmic expression of phosphorylated GSK3β ranges from about 500 to about 1500.

In on embodiment a high AQUA® score for cytoplasmic expression of phosphorylated GSK3β ranges from about 1500 to about 2500.

In on embodiment a low to intermediate AQUA® score for nuclear expression of phosphorylated CREB ranges from about 250 to 3000.

In on embodiment a high AQUA® score for nuclear expression of phosphorylated CREB ranges from about 3000 to 6000.

In on embodiment a low AQUA® score ranges for PTEN expression ranges about 200 to about 260.

In on embodiment a high AQUA® scores for PTEN expression ranges of from about 300 to about 800.

In on embodiment a low AQUA® scores for mTOR expression ranges of from about 200 to about 300.

In on embodiment a high AQUA® scores for mTOR expression ranges of from about 300 to about 800.

In on embodiment a low AQUA® scores for phosphorylated AKT expression ranges of from about 800 to about 1024.

In on embodiment an intermediate AQUA® scores for phosphorylated AKT expression ranges of from about 1024 to about 1500

In on embodiment a high AQUA® scores for phosphorylated AKT expression ranges of from about 1500 to about 3000.

### BRIEF DESCRIPTION OF THE DRAWINGS

FIG. 1: AQUA® score distribution frequency histograms for biomarker expression in the tissue samples of the GBM cohort. PTEN expression AQUA® scores obtained from analysis of the GBM cohort ranged from 123 to 2344 with a median score of 314. mTOR expression AQUA® scores ranged from 112 to 1377, with a median score of 405.

FIG. 2: Two-step unsupervised cluster analysis of PTEN AQUA® scores from the GBM cohort showing patients could be segregated into two groups, one with low PTEN expression (49% of patients) and a second with high PTEN expression (39% of patients).

FIG. 3: Kaplan-Meier survival analysis shows a significant (p=0.043) 25.5% reduction from 45.2 to 19.7% in three-year disease specific survival between patients with PTEN-high and PTEN-low expressing tumors. Median survival time is increased from 15.7 months to 24.0 months for PTEN-high expressing tumors.

FIG. 4: Two-step unsupervised cluster analysis of mTOR AQUA® scores from the GBM cohort showing patients could be segregated into two groups, one with low mTOR expression (39% of patients) and a second with high mTOR expression (49% of patients).

FIG. 5: Kaplan-Meier survival analysis shows a non-significant (p=0.206) 19.7% reduction from 38.0 to 18.3% in three-year disease specific survival between patients with mTOR-high and mTOR-low expressing tumors. Median survival time is increased from 16.2 months to 22.3 months for mTOR-high expressing tumors.

FIG. 6: Scatter plot showing linear regression of PTEN and mTOR AQUA® scores with indicated divisions based on clustering of each individual gene's protein expression value as measured by AQUA® analysis.

FIG. 7: Kaplan-Meier survival analysis for PTEN-high/mTOR-high expressing group defined in FIG. 6 showing a significant (p=0.011) 32% increase from 21.5 to 53.5% in three-year disease specific survival for the PTEN high/mTOR high expressing group. Median survival for the PTEN high/mTOR high exceeded 36 months. The other 3 groups were combined due to similarity in curve shape and median survival (when plotted separately). Comparing the high/high to all others showed a significant association with three-year disease specific survival (31.9% increase from 21.6 to 53.5% in 3-year disease-specific survival; p=0.013).

FIG. 8: AQUA® score distribution frequency histograms for biomarker expression in the tissue samples of the GBM cohort. The pmTOR expression AQUA® scores ranged from 195 to 4869 to, with a median of 710. The pAKT expression AQUA® scores obtained from analysis of the GBM cohort ranged from 606 to 3351 with a median of 1252.

FIG. 9: pAKT two-step unsupervised cluster analysis of pAKT AQUA® scores from the GBM cohort showing patients could be segregated into three groups, one with low pAKT expression (25.5% of patients); a mid pAKT expression group (31.2% of patients); and a high pAKT expression group (37.2% of patients).

FIG. 10: Kaplan-Meier survival analysis shows a significant (p=0.047) 27.2% reduction in one-year disease specific survival between pAKT high and pAKT low expressing patients.

FIG. 11: Scatterplot showing linear regression of PTEN and pAKT AQUA® scores with indicated divisions based on clustering of each individual gene's protein expression value as measured by AQUA® analysis.

FIG. 12: Kaplan-Meier survival analysis for PTEN/pAKT combined cluster expressing group as defined in FIG. 11 showing a significant (p=0.00005) 56.1% decrease from 22.2% to 78.3% in one-year disease specific survival for the PTEN-low/pAKT-high expressing group. Median survival for the PTEN-low/pAKT-high was 4.2 months Right, Kaplan-Meier analysis with all groups; Left, Kaplan-Meier analysis with groups 1-5 combined compared to group 6. Kaplan-Meier survival analysis for three-year disease-specific survival was similar in shape and curve distribution (p=0.004; data not shown).

FIG. 13: Summary of Cox proportional hazards model for one-year disease specific survival using continuous AQUA® scores showing indicated marker, hazard ratio, 95% confidence interval (95CI), p-values for each marker, and p-values for the overall indicated model (Table). Risk equation is also given based on coefficients from each marker as generated by the optimal Cox model. This equation was applied to each patient in YTMA85 to yield a risk index; distribution histogram of risk indexes is shown as well as a model for how risk would be ascertained for patients based on their risk.

FIG. 14: Summary of Cox proportional hazards model for three-year disease specific survival using categorical AQUA® scores showing indicated marker, hazard ratio, 95% confidence interval (95CI), p-values for each marker, and p-values for the overall indicated model (Table). Risk equation is also given based on coefficients from each marker as generated by the Cox model. This equation was applied to each patient in YTMA85 to yield a risk index; distribution histogram of risk indexes is shown as well as a model for how risk would be ascertained for patients based on their risk.

FIG. 15: Multiplexing AQUA® analysis differentially stains both cellular compartments and/or target genes.

FIG. 16: AQUA® score regression analysis for each indicated biomarker between redundant tissue cores from YTMA85.

FIG. 17: Kaplan-Meier survival analysis.

FIG. 18: mTOR adds to the prognosis given by PTEN.

FIG. 19: Hierarchical clustering analysis.

FIG. 20: Cox Proportional Hazards Model

FIG. 21: Results of GSK3B nuclear expression cluster analysis.

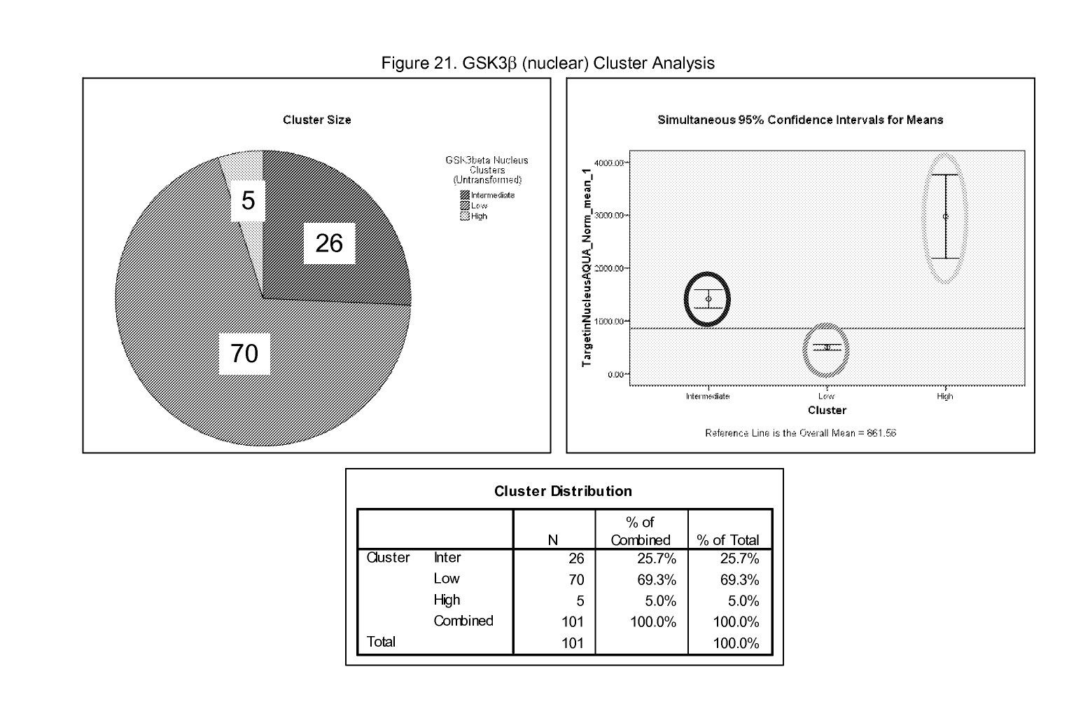

FIG. 22: Results of GSK3β (nuclear) Kaplan-Meier Survival analysis.

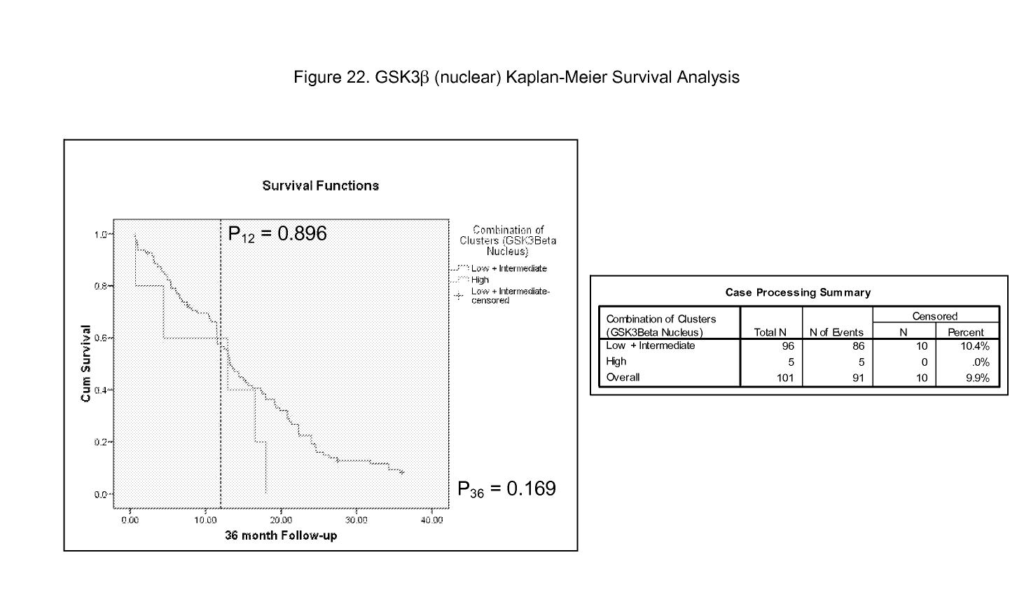

FIG. 23: Results of GSK3B cytoplasmic expression cluster analysis.

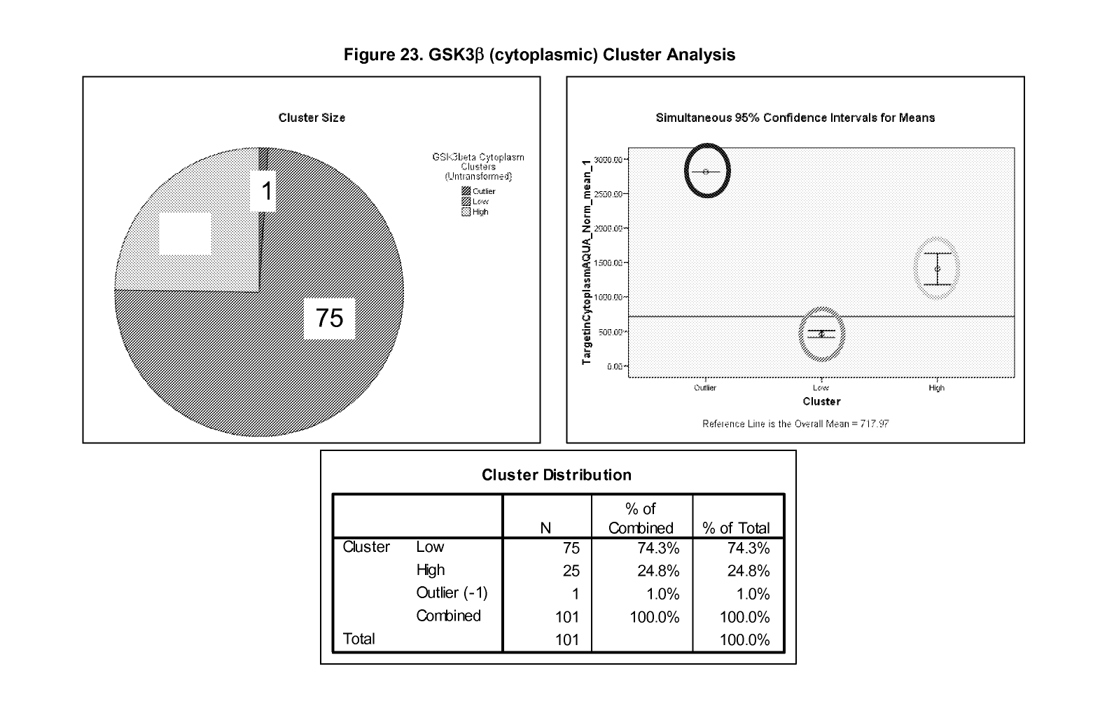

FIG. 24: Results of GSK3β (cytoplasmic) Kaplan-Meier Survival analysis.

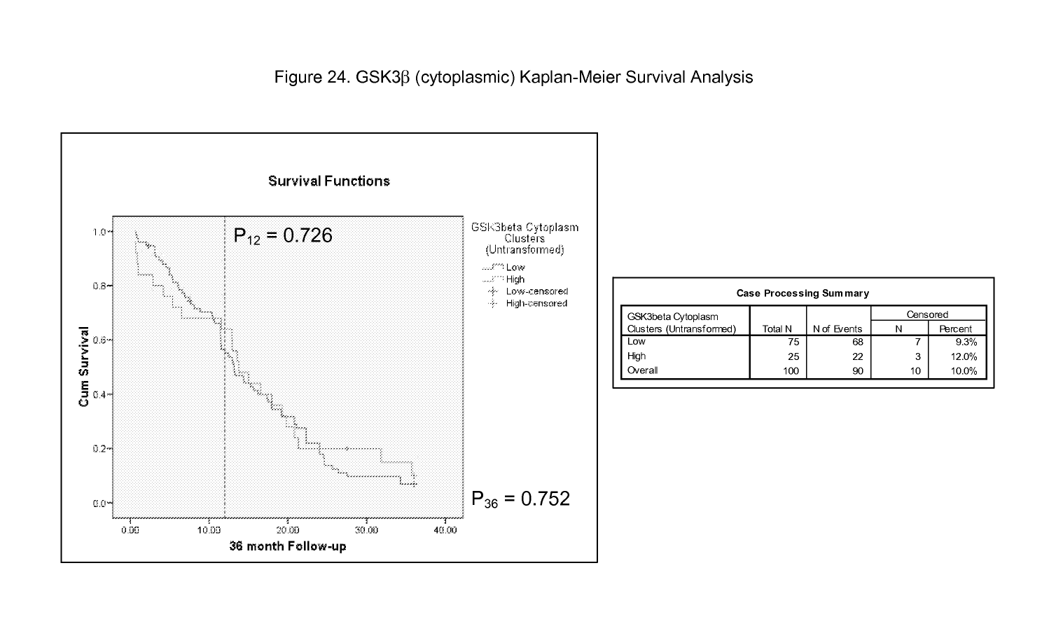

FIG. 25: Results of Phospho-GSK3β ser9 (cytoplasmic) cluster analysis.

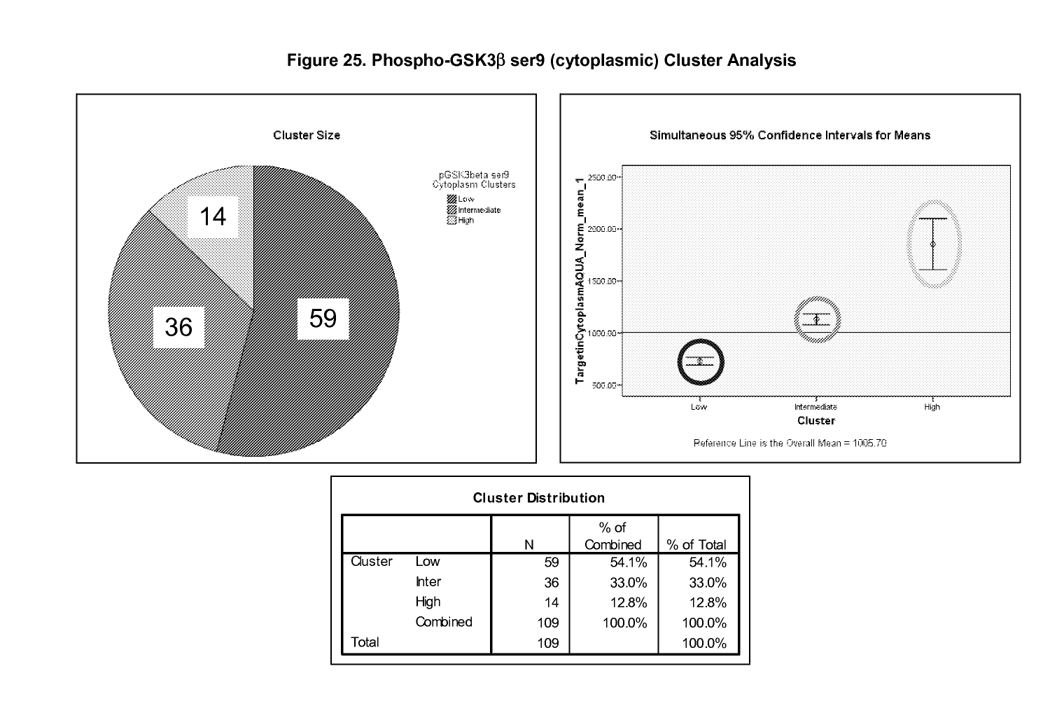

FIG. 26: Results of Phospho-GSK3β ser9 (cytoplasmic) Kaplan-Meier Survival analysis.

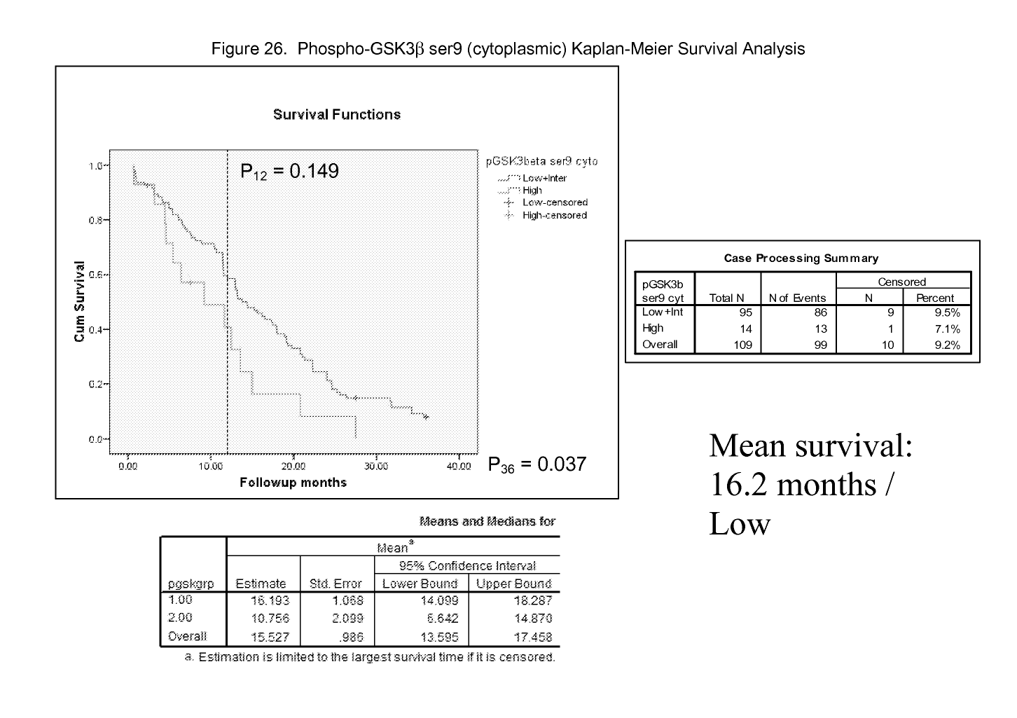

FIG. 27: Results of Phospho-S6 ser240/244 cluster analysis.

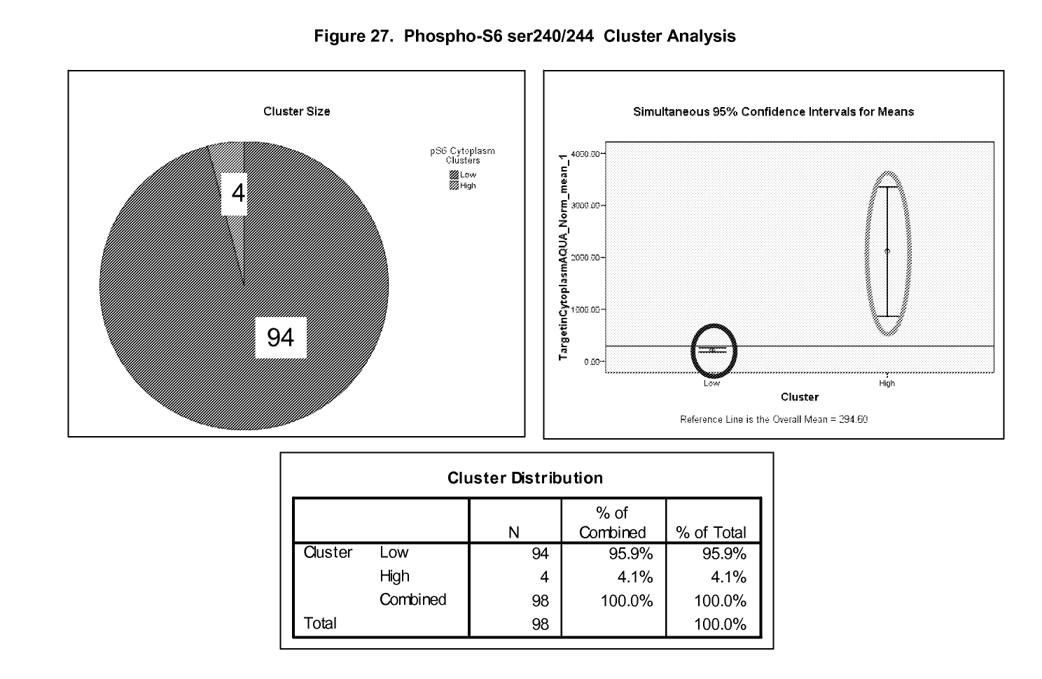

FIG. 28: Results of Phospho-CREB ser133 cluster analysis.

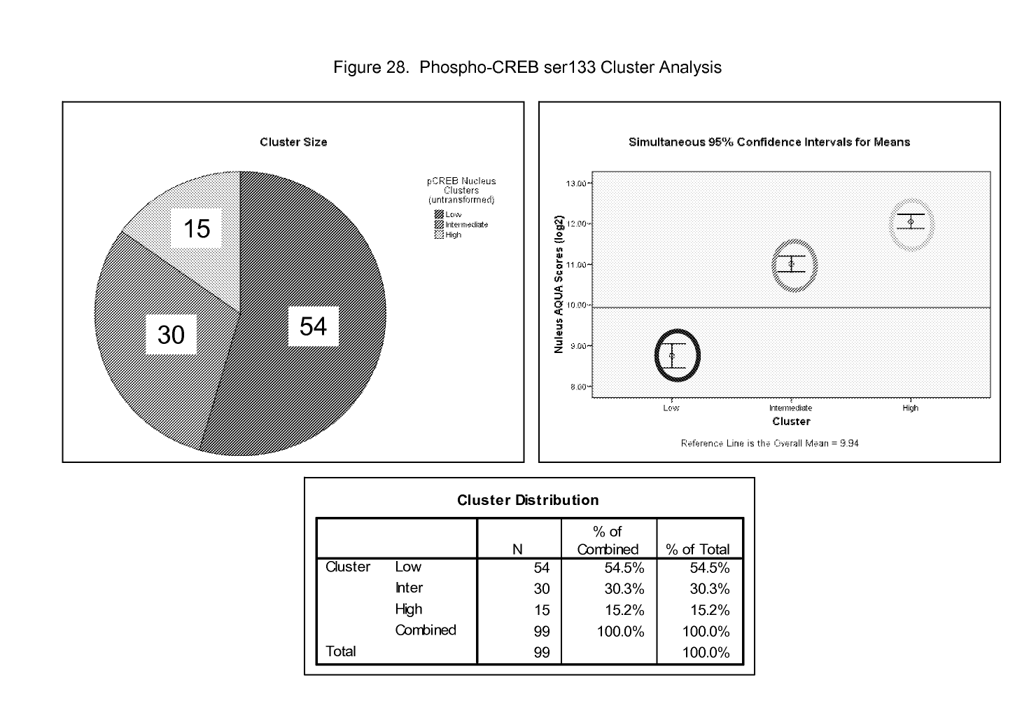

FIG. 29: Results of Phospho-CREB ser133 Kaplan-Meier Survival analysis.

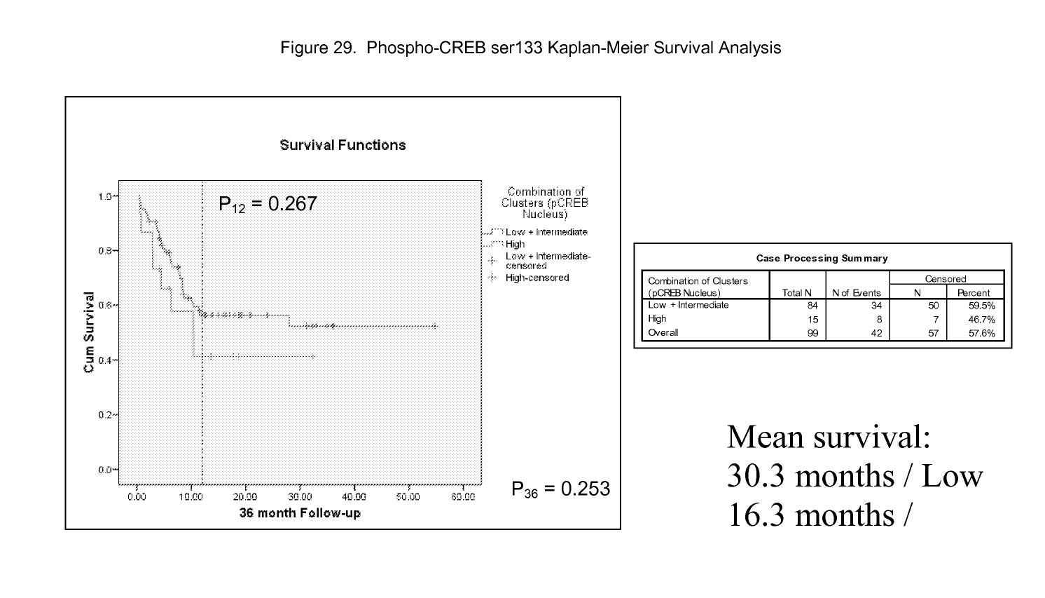

FIG. 30: The MCA's discrimination measures.

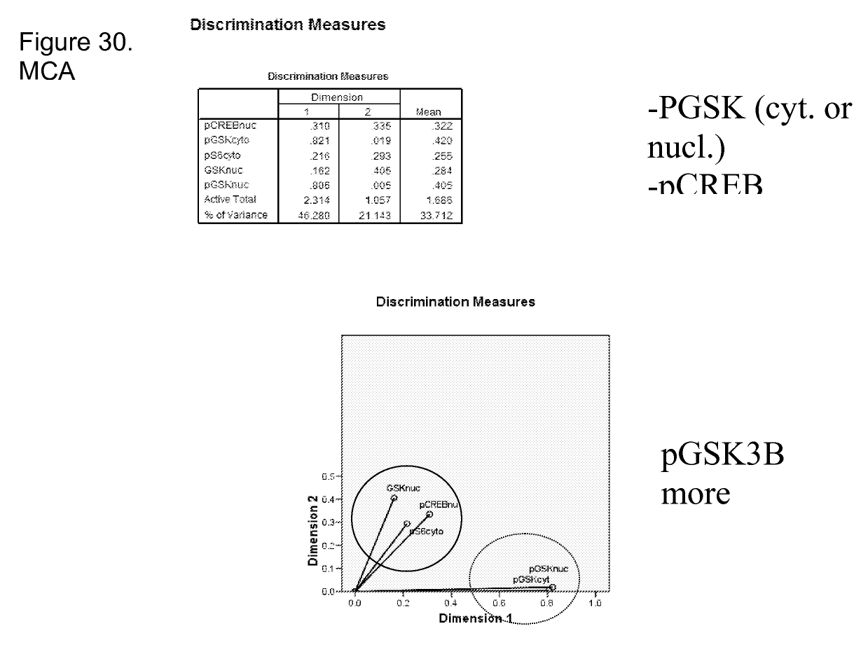

FIG. 31: The MCA (GBM markers)'s joint plot of category points.

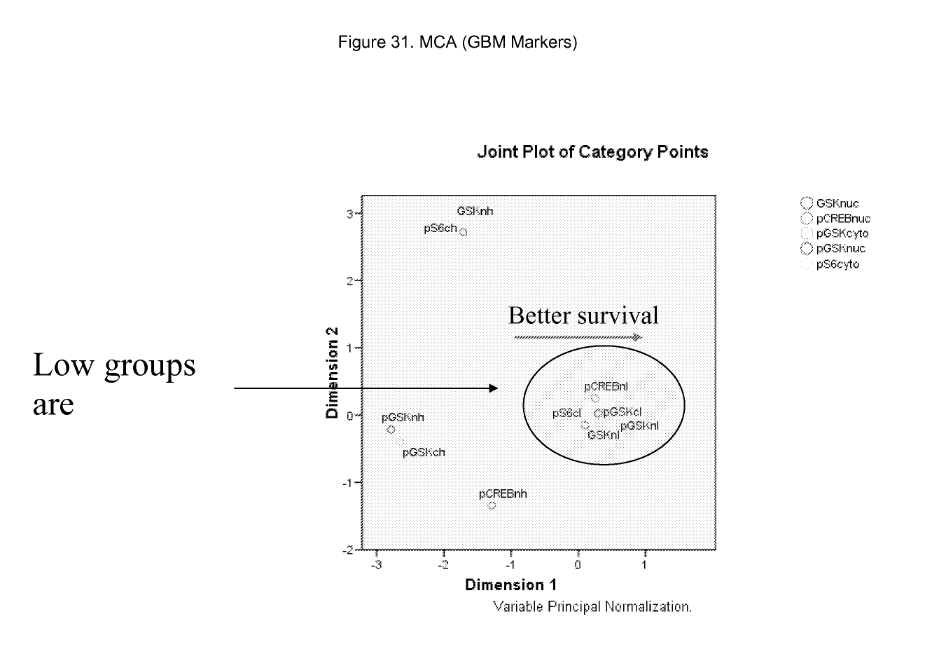

### DETAILED DESCRIPTION

In one embodiment, there is provided a method of identifying a patient suitable for treatment with a pharmaceutical inhibitor of the PI3k/AKT/mTOR pathway. The method comprises a step of assessing the relative concentration of one or more phosphorylated biomarkers in a tissue specimen obtained from the patient, wherein high levels of the one or more phosphorylated biomarkers indicates the patient is likely to benefit from treatment with the pharmaceutical inhibitor. In some embodiments the pharmaceutical inhibitor for treating a patient is selected from the group consisting of Rapamycin, Temsirolimus (Torisel), Everolimus (RAD001), AP23573, Bevacizumab, BIBW 2992, Cetuximab, Imatinib, Trastuzumab, Gefitinib, Ranibizumab, Pegaptanib, Sorafenib, Sasatinib, Sunitinib, Erlotinib, Nilotinib, Lapatinib, Panitumumab, Vandetinib, E7080, Sunitinib, Pazopanib, Enzastaurin, Cediranib, Alvocidib, Gemcitibine, Axitinib, Bosutinib, Lestartinib, Semaxanib, Vatalanib or combinations thereof. In some embodiments of the method, the patient is naïve. In some embodiments, the patient suffers from brain cancer. In some embodiments, the brain cancer is glioblastoma. In some embodiments, the pharmaceutical inhibitor is Enzastaurin. In some embodiments, the biomarkers are GSK3B, S6, CREB, PTEN, AKT, mTOR and pmTOR.

In one embodiment, there is provided a method of determining the prognosis of a patient. The method comprises a step of assessing the relative concentration of one or more phosphorylated biomarkers in a tissue specimen obtained from the patient, wherein high levels of the one or more phosphorylated biomarkers indicates the patient has a relatively poor prognosis and wherein low levels of one or more phosphorylated biomarkers indicates the patient has a relatively better prognosis. In some embodiments of the method, the patient is naïve. In another embodiments, the patient is undergoing a treatment with an inhibitor of the PI3k/AKT/mTOR pathway. In some embodiments, the patient suffers from brain cancer. In some embodiments, the brain cancer is glioblastoma. In some embodiments, the pharmaceutical inhibitor is Enzastaurin. In some embodiments, the biomarkers are GSK3B, S6, or CREB.

In some embodiments of the method, the patient suffers from cancer. In some embodiments the cancer is selected from a group consisting of: brain cancers, prostate cancers, breast cancers, colorectal cancers and pancreatic cancers and non small cell lung cancer (NSCLC). In some preferred embodiments of the method, the patient suffers from a brain cancer. In some embodiments, the brain cancer is glioblastoma. In some embodiments, the pharmaceutical inhibitor is Enzastaurin. In some embodiments, the biomarkers are GSK3B, S6, or CREB. In some embodiments, the subcellular compartment is cytoplasm. In some embodiments, the stain that specifically labels the subcellular compartment comprises a stain for GFAP. In some embodiments of the method, in step b), a high resolution image of each of the first, the second and the third stain in the tissue sample is obtained using a microscope.

In one embodiment, there is provides a kit, which comprises

a) a first stain specific for a phosphorylated biomarker;

b) a second stain specific for a first subcellular compartment of a cell; and

c) instructions for using the kit.

In some embodiments of the kit, the biomarkers are GSK3B, S6, or CREB. In some embodiments, the second stain is for GFAP. In some embodiments, the kit further comprises a third stain specific for a second subcellular compartment of a cell.

Inventors have found that relative concentrations of phosphorylated markers can be determined in tissue samples using AQUA® analysis.

A retrospective glioblastoma multiforme cohort of 115 patients was evaluated by quantitative immunofluoresence using AQUA® analysis for protein levels of phosphoCREB ser133, phosphoS6 ser240/244, phosphoGSK3B ser9 and total GSK3B expression in formalin fixed paraffin embedded (FFPE) tissue specimens.

Inventors have discovered that high expression of phosphor-GSK3B in tissue specimens is significantly associated with worse patient outcome or poor prognosis whereas low expression of phospho-GSK3B in tissue specimens is significantly associated with better patient outcome or better prognosis.

Similarly the inventors identified a trend in high expression of phospho-Creb in tissue specimens is associated with poor prognosis whereas low expression of phospho-Creb is associated with better prognosis.

Inventors have discovered a tissue based assay method for determining levels of a biomarker(s) selected from the group consisting of: GSK3β, pGSK3β ser9, pS6ser240/244 and pCREBser133 in tissue specimens. Furthermore inventors have shown a method of determining prognosis of a patient based upon the assesment of phosphorylated biomarker(s) levels, the markers selected from the group consisting of pGSK3β ser9, pS6ser240/244 and pCREBser133 in a tissue specimen wherein low levels of a phosphorylated marker is associated with relatively better survival and high levels of a phosphorylated marker is associated with relatively poor survival.

The method can be used for identifying a patient for a treatment in which the treatment blocks signaling through the PI3k, AKT, mTOR pathway. The method can be used for identifying a patient for treatment with Enzastaurin, particularly a patient which may particularly benefit from such treatment.

Furthermore the invention pertains to a kit comprising: an immunoreagent for detecting, a biomarker, GBM tissue, and a reagent for detecting nuclei in a tissue specimen, secondary detection reagents and instructions for carrying out an immunoassay in tissue for determining the relative quantity of the phosphorylated biomarker. The biomarker may be GSK3β, pGSK3β ser9, pS6ser240/244 and pCREBser133 and the immunoreagent for detecting the biomarker may be an antibody specific for the biomarker.

The present invention is further described by reference to the following examples which are illustrative and not limiting of the invention.

In one embodiment, there is provided a method of determining a prognosis of a patient. In one embodiment, the method comprises quantitatively assessing the concentration of one or more protein biomarkers, including PTEN and/or mTOR, in a tissue specimen obtained from the patient wherein high levels of PTEN and mTOR indicate the patient has a relatively good prognosis and wherein low levels of PTEN or mTOR indicate the patient has a relatively poor prognosis.

In another embodiment, the method comprises quantitatively assessing the concentration of pAKT or pmTOR protein biomarker in a tissue specimen obtained from the patient, wherein high levels of pAKT indicate the patient has a relatively poor prognosis and wherein low levels of pAKT indicate the patient has a relatively good prognosis.

In these embodiments, the patient suffers from brain cancer such as glioblastoma. The patient being evaluated may be naïve or undergoing treatment with an inhibitor of the PI3 kinase/AKT/mTOR pathway. The inhibitor may be Enzastaurin or rapamycin or other mTOR inhibitors, optionally combined with temozolomide and/or radiation.

In one embodiment, there is provided a method of determining the prognosis of a patient. The method comprises quantitatively assessing the concentration of PTEN and mTOR protein biomarkers in a tissue specimen obtained from the patient, wherein high PTEN and high mTOR protein expression levels indicates the patient has a relatively good prognosis and wherein low PTEN and low mTOR, high PTEN and low mTOR, low PTEN and high mTOR levels of protein expression indicates the patient has a relatively poor prognosis.

In another embodiment, there is provided a method of determining a prognosis of a patient, which comprises quantitatively assessing the concentration of PTEN and pAKT protein biomarkers in a tissue specimen obtained from the patient, wherein high pAKT and low PTEN protein expression levels indicates the patient has a relatively very poor prognosis compared to low PTEN and low pAKT; low PTEN and medium pAKT; high PTEN and low pAKT; high PTEN and medium pAKT; and high PTEN and high pAKT protein expression levels.

In these embodiments, the patient suffers from brain cancer such as glioblastoma. The patient being evaluated may be naïve or undergoing treatment with an inhibitor of the PI3 kinase/AKT/mTOR pathway. The inhibitor may be Enzastaurin or rapamycin or other mTOR inhibitors, optionally combined with temozolomide and/or radiation.

In one embodiment, there is provided a method of determining the prognosis of a patient by quantitatively assessing the concentration of one or more biomarkers in a tissue sample. The method comprises:
a) incubating the tissue sample with a first stain that specifically labels a first marker defined subcellular compartment, a second stain that specifically labels a second marker defined subcellular compartment and a third stain that specifically labels the biomarker;
b) obtaining a high resolution image of each of the first, the second and the third stain in the tissue sample;
c) assigning a pixel of the image to a first compartment based on the first stain intensity; a second compartment based on the second stain intensity; or to neither a first nor second compartment;
d) measuring the intensity of the third stain in each of the pixels assigned to either the first or the second compartment or both;
e) determining a staining score indicative of the concentration of the biomarker in the first or the second compartment or both; and f) plotting the biomarker concentration in relationship to a second biomarker concentration thereby providing a determination of the patient's prognosis.

The tissue sample may be obtained from a patient suffering from brain cancer such as glioblastoma.

In one embodiment, the biomarker may be PTEN, and a second biomarker may be mTOR or pAKT.

In some embodiments, high expression of PTEN together with high expression of mTOR in a tissue sample is indicative or relatively good prognosis. In some embodiments, low expression of PTEN together with high expression of pAKT in a tissue sample is indicative of relatively poor prognosis.

In some embodiments, a subcellular compartment is cytoplasm, the stain that specifically labels the subcellular compartment comprises a stain for GFAP.

In some embodiment, there is provided a kit comprising: a) a first stain specific for PTEN; b) a second stain specific for a first subcellular compartment of a cell; and c) instructions for using the kit. In the kit, the second stain is for GFAP. The kit may further comprise a specific stain for mTOR. The kit may still further comprise a third stain specific for a second subcellular compartment of a cell.

In another embodiment, there is provided a kit which comprises: a) a first stain specific for mTOR; b) a second stain specific for a first subcellular compartment of a cell; and c) instructions for using the kit. In the kit, the second stain is for GFAP. The kit may further comprise a third stain specific for a second subcellular compartment of a cell.

In one embodiment, there is provided a kit which comprises: a) a first stain specific for pmTOR; b) a second stain specific for a first subcellular compartment of a cell; and c) instructions for using the kit. In the kit, the second stain is for GFAP. The kit may further comprise a third stain specific for a second subcellular compartment of a cell.

In one embodiment, there is provided a kit which comprises: a) a first stain specific for pAKT; b) a second stain specific for a first subcellular compartment of a cell; and c) instructions for using the kit. In the kit, the second stain is for GFAP. The kit may further comprise a third stain specific for a second subcellular compartment of a cell.

In one embodiment, there is provided a method of identifying a patient suitable for treatment with a pharmaceutical inhibitor of the PI3k/AKT/mTOR pathway. The method comprises: quantitatively assessing the concentration of one or more biomarkers, or phosphorylated forms thereof, in a tissue specimen obtained from the patient wherein high levels of one or more biomarkers indicate the patient is likely to benefit from treatment with the pharmaceutical inhibitor. In some embodiments, the patients suffer from brain cancer such as glioblastoma. In some embodiments, the pharmaceutical inhibitor is Enzastaurin or rapamycin. In some embodiments, the biomarkers are chosen from the group consisting of PTEN and mTOR. In some embodiments, the patient may be naïve.

In one embodiment, it is provided a method of determining the prognosis or relative risk of a patient, comprising quantitatively assessing the concentration of GSK3B, S6, CREB, PTEN, AKT and mTOR, protein biomarkers, or phosphorylated forms thereof, in a tissue specimen obtained from the patient, wherein expression or AQUA® score of each biomarker on a continuous scale is put into a Cox regression model for continuous variables resulting in a calculation of overall patient risk.

In another embodiment, there is provided a method of determining the prognosis or relative risk of a patient, comprising quantitatively assessing the concentration of GSK3B, S6, CREB, PTEN, AKT and mTOR protein biomarkers, or phosphorylated forms thereof, in a tissue specimen obtained from the patient, wherein expression or AQUA® score of each biomarker is first categorized into low and high based on optimal univariate cutpoints, then applied to a Cox regression model for categorical variables resulting in a calculation of overall patient risk.

In some embodiments, the prognosis of relative risk is for a one-year or a three-year period.

In some embodiments, the relative risk is evaluated in a model wherein one or more of the four biomarkers contribute. In some embodiments, PTEN, pAKT, mTOR, or combination thereof contribute more significantly than the others.

Inventors have found that quantitative assessment of PTEN or mTOR protein in tissue sections can be done using AQUA® analysis which showed a continuous scale of expression in tumor specimens from patients with (GBM).

Inventors have discovered that low expression of PTEN in tissue specimens is significantly associated with worse patient outcome or poor prognosis whereas high expression of PTEN in tissue specimens is significantly associated with relatively better patient outcome or better prognosis. Patients with high PTEN expression showed an 8.4 month improved median three-year disease specific survival rate from 15.6 months to 24.0 months (19.7% to 43.2% survival) and this was significant at the 10% level (p=0.062).

Similarly the inventors identified a trend in that high expression of mTOR in tissue specimens is associated with improved survival. Patients with high mTOR expression showed a 6.1 month improved median three-year disease specific survival rate from 16.2 to 22.3 months (18.3% to 39.8% survival), but this was not significant (p=0.17). There was not a significant association between continuous mTOR AQUA® scores and survival.

Furthermore the inventors took advantage of the continuous nature of AQUA scores, multiplexing PTEN and mTOR AQUA® data to produce a combined patient outcome assessment. Using unsupervised clustering cutpoints for PTEN and mTOR expression data, four groups representing low/low, high/low, low/high, and high/high PTEN/mTOR expression respectively were created. The median disease free survival for the high/high group exceeded 36 months (53.5% disease specific survival at 36 months). This association was significant at the 10% level (p=0.082).

Comparing the survival of patients with high/high PTEN, mTOR expression to all others showed a significant association with three-year disease specific survival (31.9% increase from 21.6 to 53.5% in 3-year disease-specific survival; p=0.013).

Inventors demonstrated that the combined prognostic assay utilizing both biomarkers PTEN and mTOR as determined by AQUA® analysis better predicts for a group of patients that do relatively well than as predicted by PTEN and/or mTOR alone. Considering overall median survival rates for GBM are between 12 and 15 months, identification of a population of patients whose median survival exceeds 36 months is of large potential value to both patients and physicians.

Inventors have discovered a tissue based assay method for determining quantitative levels (on a continuous scale) of biomarker(s) PTEN and mTOR in tissue specimens. Furthermore inventors have shown a method of determining prognosis of a patient based upon the assessment of PTEN and mTOR biomarker(s) levels in a tissue specimen wherein high levels of PTEN and/or PTEN along with mTOR are associated with relatively better survival.

The method can be used for identifying a patient for a treatment in which the treatment blocks signaling through the PI3k, AKT, mTOR pathway. The method can be used for identifying a patient for treatment with Enzastaurin, particularly a patient which may particularly benefit from such treatment.

Furthermore the invention pertains to a kit comprising: an immunoreagent for detecting, a biomarker, GBM tissue, and a reagent for detecting nuclei in a tissue specimen, secondary detection reagents and instructions for carrying out an immunoassay in tissue for determining the quantity of the phosphorylated biomarker. The biomarker may be PTEN and mTOR and the immunoreagent for detecting the biomarker may be an antibody specific for the biomarker.

The present invention is further described by reference to the following examples which are illustrative and not limiting of the invention.

### GS3K/S6/CREB Examples

### Cohort information

The HistoRx YTMA85 brain cancer cohort contains 183 histospots with 2× redundancy. The mean follow-up time is 25.6 months. There were 80 cases with DOD (dead of disease) status, whose average age at the time of death was 51.2 years. The majority, 76%, of the cases were in localized nodal stage and 64% were glioblastomas (Table 1). 19% of the patients had astrocytomas and the remainder of the patients had other types of brain cancer which are listed under “tumor type” (Table 1). The correlation of biomarker expression with survival analysis was evaluated only for patients with glioblastomas.

TABLE 1
Description of Brain cancer Cohort
BRAIN CANCER COHORT

| Total | | | | | | | | | | | | | |
|---|---|---|---|---|---|---|---|---|---|---|---|---|---|
| Number of | | | DOD Status⁽²⁾ | | | | | | | | | | |
| Specimens⁽¹⁾ | Follow-up (months) | | | overall | | Age (Years) | | Nodal Stage⁽²⁾ | | | Tumor Type | | |
| 183 | Mean | 25.6 | Dead With | 80 | 53.0% | Mean | 51.2 | Reg, | 2 | 1.1% | Astrocytoma | 34 | 18.58% |
| | | | Disease | | | | | DirEx | | | | | |
| | Median | 16.2 | Censored⁽²⁾ | 71 | 47.0% | Median | 52.4 | Distant | 2 | 1.1% | Oligodendroglioma | 3 | 1.64% |
| | Min | 0.6 | | | | Min | 0.8 | Localized | 140 | 76.5% | Oligoastrocytoma | 2 | 1.09% |
| | Max | 216.7 | | | | Max | 86.3 | Reg NOS | 7 | 3.8% | Glioblastoma | 118 | 64.48% |
| | Std | 33.7 | | | | Std | 18.1 | | | | Normal controls | 12 | 6.56% |
| | N | 151 | | | | N | 169 | | | | Cell Lines | 14 | 7.65% |

Note:
Information on age at diagnosis was not provided.
⁽¹⁾Data had a 2x redundancy—approximately 2 cores per specimen available—total of at least 183 cores.
⁽²⁾Percentages were calculated based upon N = 151. DOD & nodal stage status not available for 6 specimens.

### Staining Protocol

Paraffin sections were deparaffinized in xylene and hydrated and then put in Tris EDTA buffer PT Module™ Buffer 4 (100× Tris EDTA Buffer, pH 9.0) TA-050-PM4X (Lab Vision Corp, Fremont Calif.) for antigen retrieval. Sections were then rinsed once in 1×TBS Tween (Lab Vision, Fremont, Calif.) for 5 minutes and incubated in peroxidase block (Biocare Medical, Concord, Calif.) for 15 min followed by a rinse in 1×TBS Tween for 5 min. Sections were blocked using Background Sniper (Biocare Medical, Newport Beach, Calif.) for 15 min. Sections were incubated with the primary antibody cocktail: rabbit anti-biomarker antibody and mouse anti-GFAP (DAKO, lot #M076101-2 at a 1:100 concentration) diluted in DaVinci Green (Biocare Medical, Newport Beach, Calif.) for 1 hours at room temp. In this study rabbit anti-biomarker antibodies included: total GSK3β (Cell Signaling #9315 at 1:100 dilution), pGSK3β ser9 (Cell Signaling #9336 at 1:10 dilution), pS6ser240/244 (Cell Signaling #2215 at 1:500 dilution), and pCREBser133 (Cell signaling #9198 at 1:10 dilution). Following three 5 min. rinses in 1×TBS Tween, slides were incubated in secondary antibody cocktail of goat anti-rabbit EnVision (DAKO, prepared per manufacturer's instructions) and goat anti-mouse Alexa Fluor 555 (Invitrogen A21429 diluted 1:200 into the EnVision) for 30 minutes in the dark, rinsed and then treated with Cy5 tyramide, diluted 1:50 in amplification buffer (Perkin Elmer SAT705A) for 10 min. room temperature in the dark, mounted with Prolong anti-fade with DAPI (Invitrogen, Carlsbad Calif.) and allowed to dry overnight.

Each stained specimen was imaged using a PM-2000™ system (HistoRx, New Haven Conn.) at 20× magnification. A board-certified pathologist reviewed an H&E stained serial section of the glioblastoma cohort to confirm tumor tissue presence in the samples. Images were evaluated for quality prior to analysis as described in co-pending U.S. Application 60/954,303. AQUA® analysis of the biomarkers was conducted and the biomarkers are quantified within cytoplasmic and nuclear compartments as described in Camp et al 2002 Nature Medicine 8(11)1323-1327.

### Results

Staining and AQUA® analysis:

### Total GSK3B:

Staining was cytoplasmic and nuclear.

Statistics^{a}
TargetinNucleusAQUA_Norm_mean_1

| | | |
|---|---|---|
| N | Valid | 102 |
| | Missing | 0 |
| Mean | | 856.6621 |
| Median | | 606.6550 |
| Std. Deviation | | 671.62276 |
| Skewness | | 1.900 |
| Std. Error of Skewness | | .239 |
| Minimum | | 139.04 |
| Maximum | | 3642.43 |

^{a}Marker = GSK3beta

Statistics^{a}
TargetinCytoplasmAQUA_Norm_mean_1

| | | |
|---|---|---|
| N | Valid | 102 |
| | Missing | 0 |
| Mean | | 713.9447 |
| Median | | 542.1519 |
| Std. Deviation | | 529.03665 |
| Skewness | | 1.737 |
| Std. Error of Skewness | | .239 |
| Minimum | | 120.01 |
| Maximum | | 2816.42 |

^{a}Marker = GSK3beta

Phospho-GSK3B ser9:
Staining was cytoplasmic and nuclear.

Statistics^{a}
TargetinNucleusAQUA_Norm_mean_1

| | | |
|---|---|---|
| N | Valid | 110 |
| | Missing | 0 |
| Mean | | 1074.4978 |
| Median | | 948.1095 |
| Std. Deviation | | 480.98916 |
| Skewness | | 1.970 |
| Std. Error of Skewness | | .230 |
| Minimum | | 525.91 |
| Maximum | | 3011.60 |

^{a}Marker = pGSK3beta

Statistics^{a}
TargetinCytoplasmAQUA_Norm_mean_1

| | | |
|---|---|---|
| N | Valid | 110 |
| | Missing | 0 |
| Mean | | 1004.3132 |
| Median | | 881.4187 |
| Std. Deviation | | 406.11599 |
| Skewness | | 1.570 |
| Std. Error of Skewness | | .230 |
| Minimum | | 386.88 |
| Maximum | | 2621.30 |

^{a}Marker = pGSK3beta

Phospho-S6 ser240/244:
Staining was primarily cytoplasmic.

Statistics^{a}
TargetinCytoplasmAQUA_Norm_mean_1

| | | |
|---|---|---|
| N | Valid | 99 |
| | Missing | 0 |
| Mean | | 292.8274 |
| Median | | 143.9676 |
| Std. Deviation | | 423.96267 |
| Skewness | | 4.060 |
| Std. Error of Skewness | | .243 |
| Minimum | | 45.85 |
| Maximum | | 2730.74 |

^{a}Marker = pS6ser240-244

Phospho-CREB ser133:
Staining was nuclear.

Statistics^{a}
TargetinNucleusAQUA_Norm_mean_1

| | | |
|---|---|---|
| N | Valid | 100 |
| | Missing | 0 |
| Mean | | 1568.7719 |
| Median | | 1030.9511 |
| Std. Deviation | | 1444.419 |
| Skewness | | 1.103 |
| Std. Error of Skewness | | .241 |
| Minimum | | 122.19 |
| Maximum | | 5879.37 |

^{a}Marker = pCREBser133

### Clustering Analysis

AQUA® score results for each marker across the GBM cohort were analyzed by a two step unsupervised clustering algorithm.

### GSK3B:

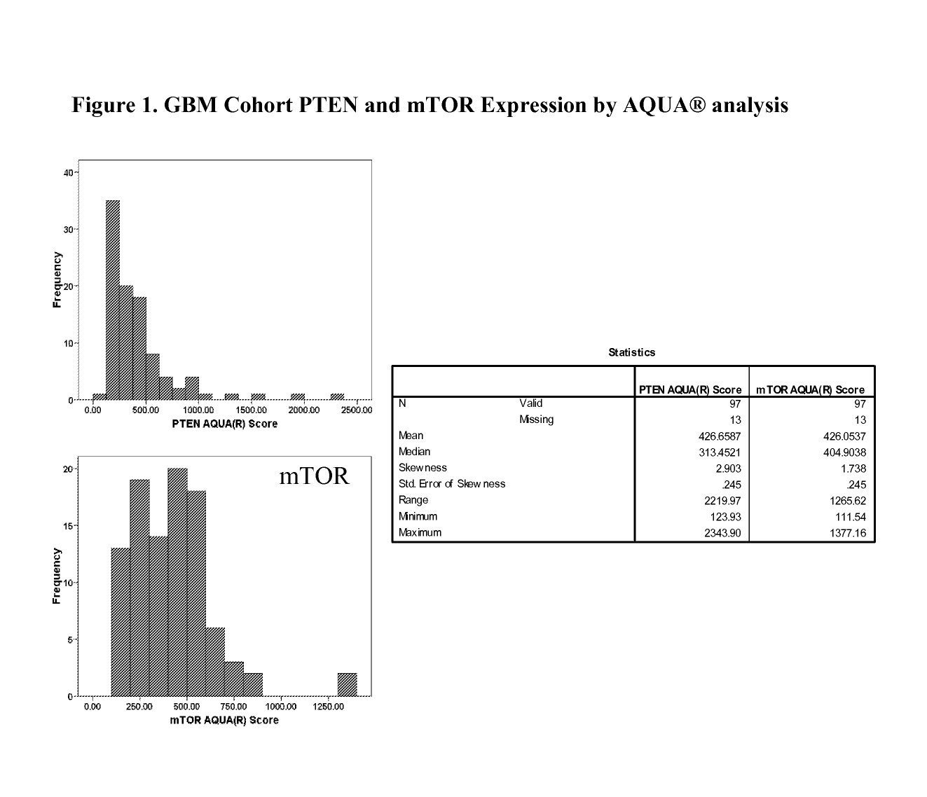

FIG. 1 shows the results of cluster analysis of GSK3B nuclear expression. Three clusters were identified characterized by low (70%), medium (25%), and high (5%) GSK3B nuclear expression.

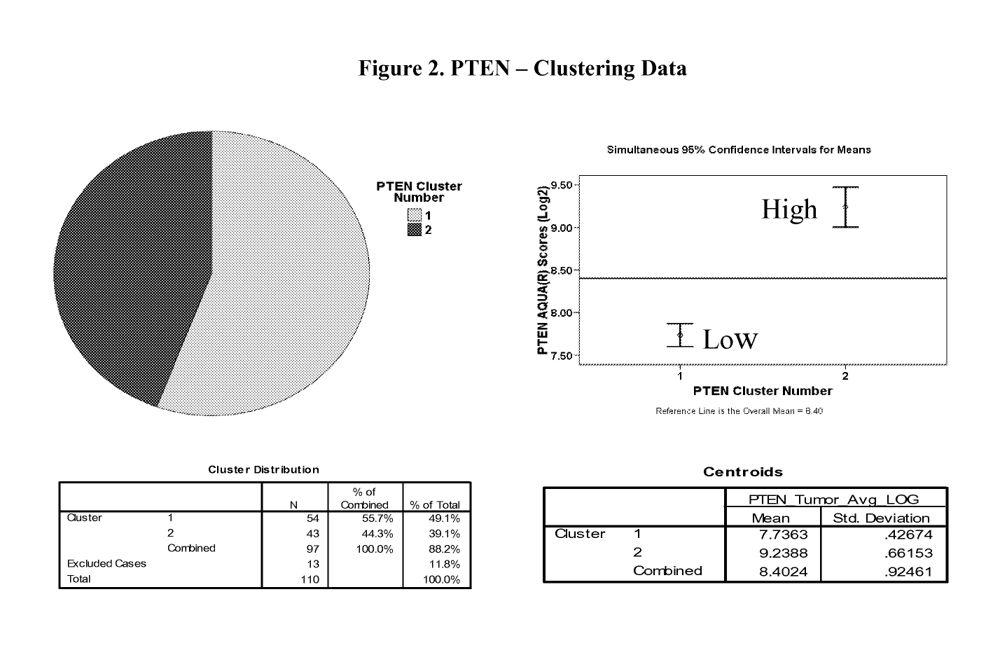

By Kaplan-Meier survival analysis, high nuclear expression of GSK3B was associated with poor survival, although this finding was not statistically significant for this cohort FIG. 2.

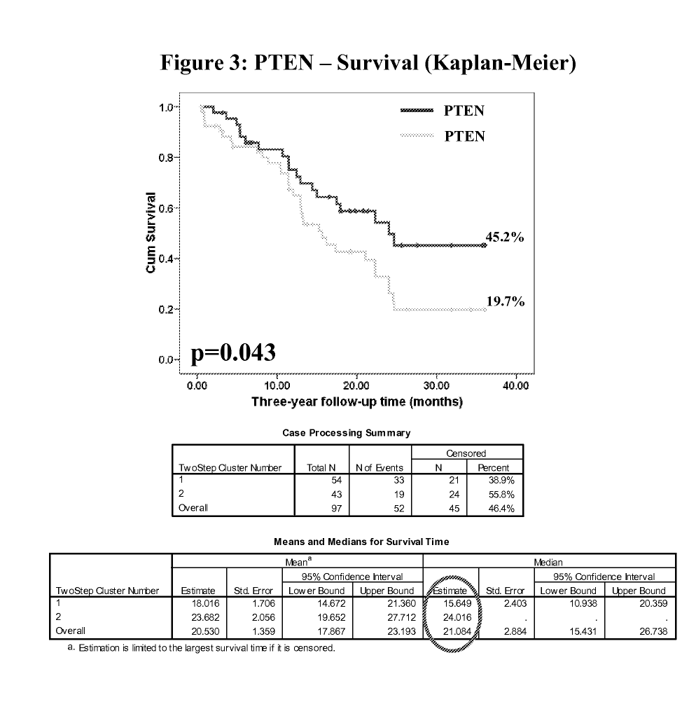

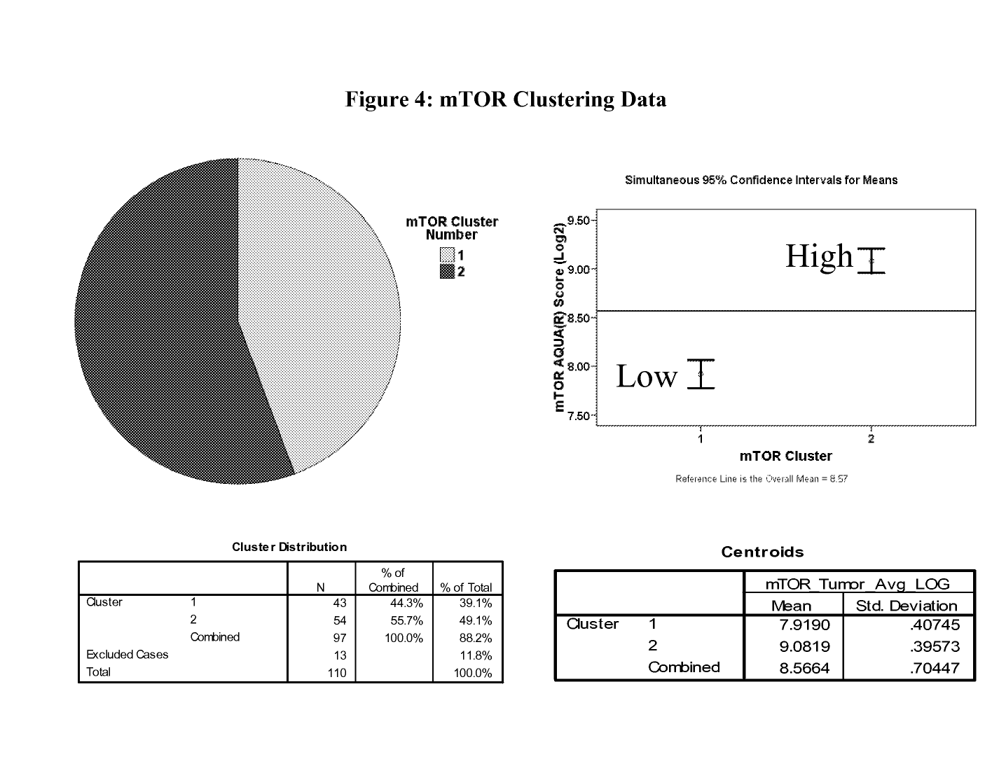

FIG. 3 shows the results of cluster analysis of GSK3B cytoplasmic expression. Essentially two clusters were identified characterized by low (75%) and high (25%) GSK3B cytoplasmic expression. By Kaplan-Meier survival analysis cytoplasmic expression of GSK3B did not significantly affect patient survival FIG. 4.

### Phospho-GSK3B:

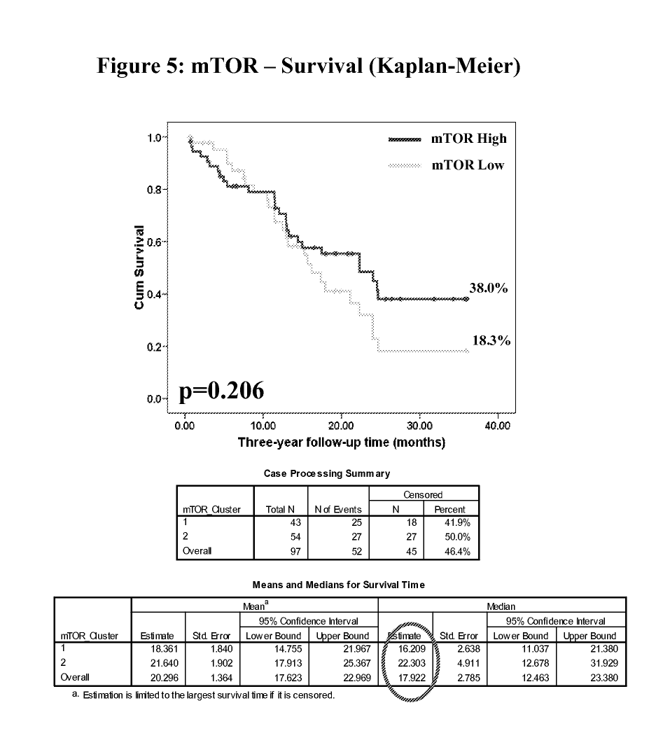

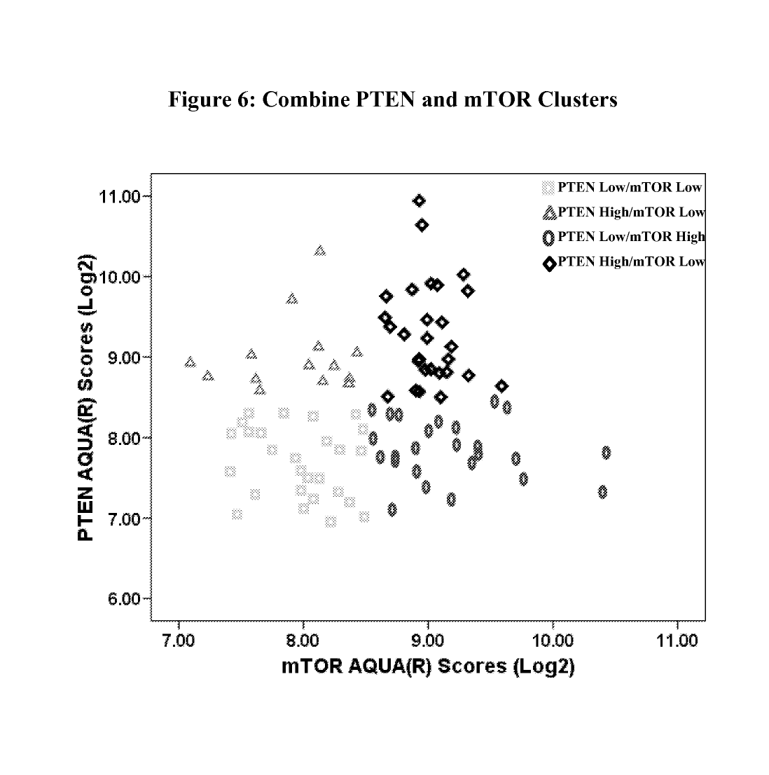

Cluster analysis of pGSK3B expression identified 3 clusters characterized by low (54%), medium (33%) and high (13%) pGSK3b cytoplasmic expression (FIG. 5). By Kaplan-Meier analysis pGSK3B expression was statistically significantly associated with survival. Patients whose tumors had low pGSK3B expression had a mean survival of 16.2 months whereas patients whose tumors had high pGSK3B expression had a mean survival of only 10.8 months (FIG. 6).

Phospho-S6 ser240/244:

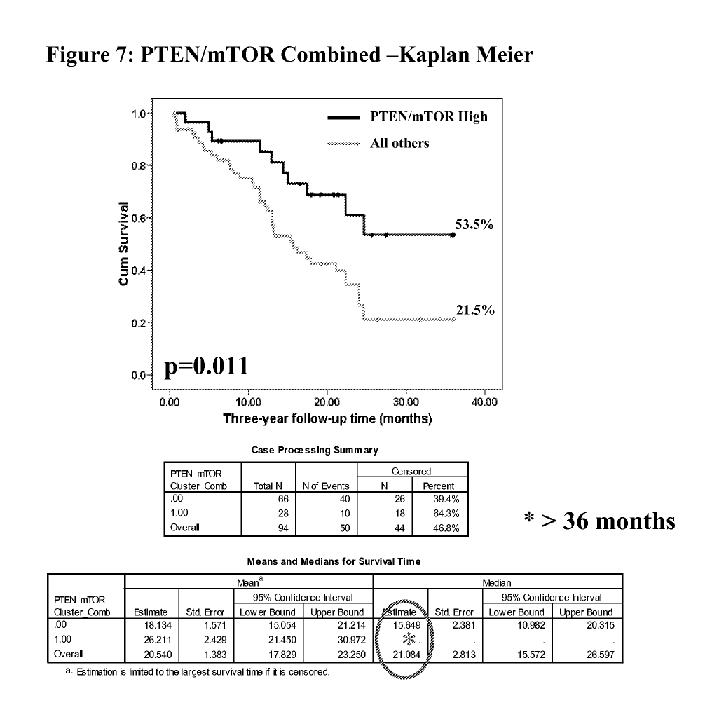

Cluster analysis of pS6ser240/244 expression identified two groups characterized by low (96%) and high (4%) pS6 expression (FIG. 7). Kaplan Meier analysis did not find a significant association of pS6 expression and survival, however there were a limited number of high expressing patients in this cohort.

Phospho-CREB ser133:

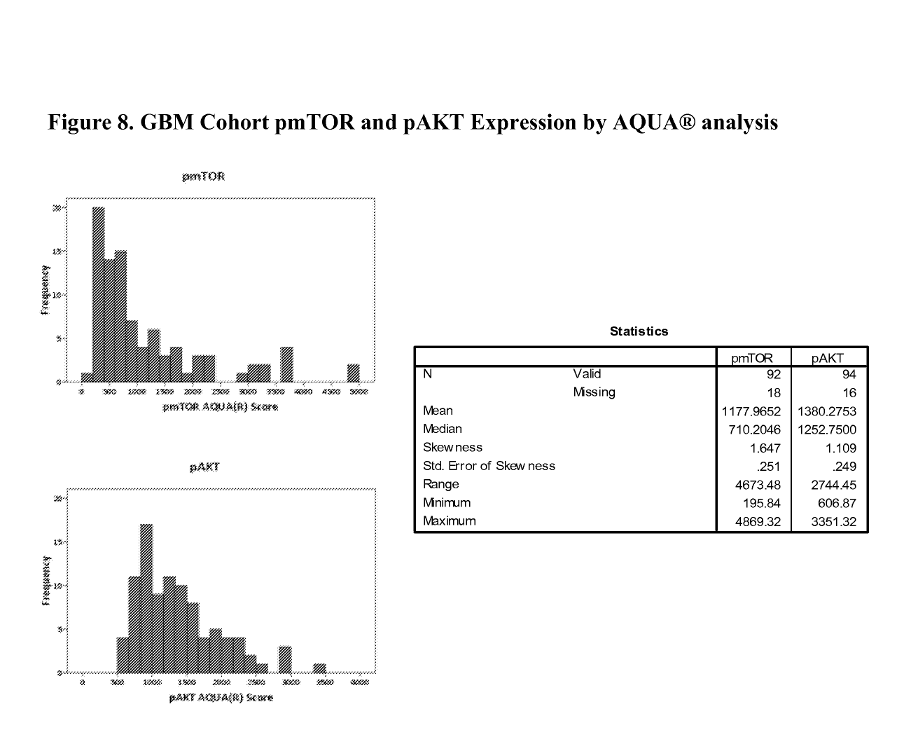

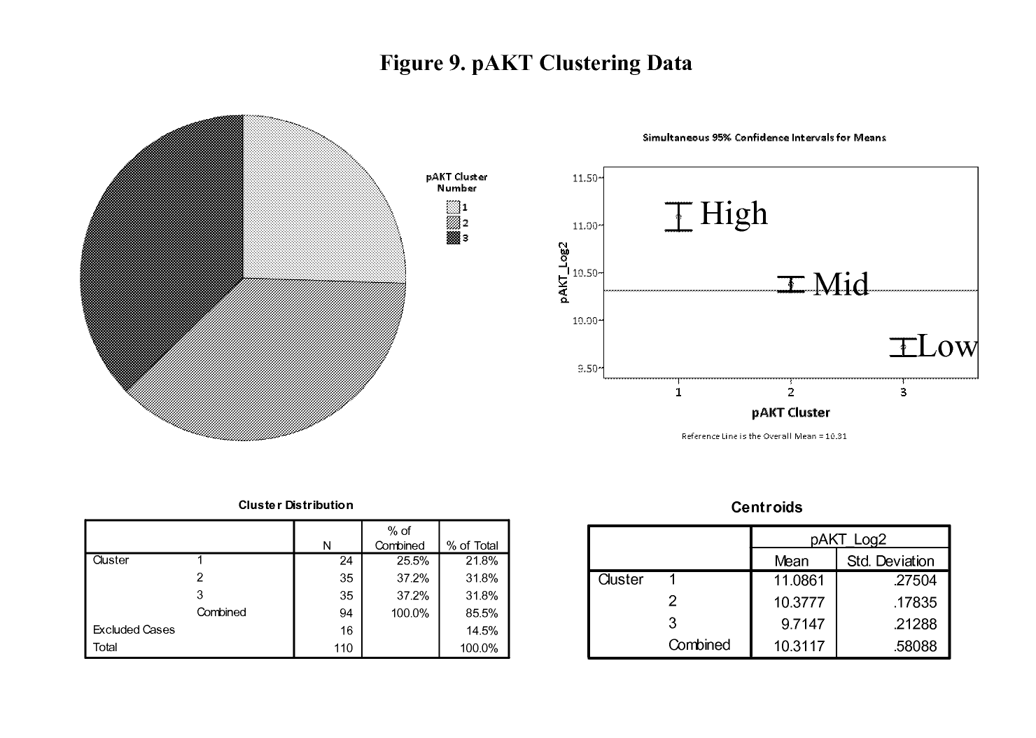

Cluster analysis of pCREBser133 expression identified three groups characterized by low (55%), medium (30%) and high (15%) expression (FIG. 8). Kaplan-Meier analysis identified a trend by which high expression of pCREBser133 was associated with worse survival where as low and medium expression was associated with better survival. Patients whose tumors had low expression of pCREB had a mean survival of 30.3 months whereas patients whose tumors had high expression of pCREB had a mean survival of only 16.3 months (FIG. 9).

TABLE 2
Summary of Survival Analysis.

| | | KM | KM | |
|---|---|---|---|---|
| | | p-value | p-value | |
| Biomarker | Compartment | at 12 mo | at 36 mo | Survival |
| GSK3B | nuclear | 0.896 | 0.196 | |
| | cytoplasmic | 0.726 | 0.752 | |
| pGSK3B | cytoplasmic | 0.149 | 0.037 | Low = 16.2 mo |
| | | | | High = 10.8 mo |
| pS6 | cytoplasmic | 0.539 | 0.791 | |
| ser240/244 | | | | |
| pCREB | nuclear | 0.267 | 0.259 | Low = 30.3 mo |
| ser133 | | | | High = 16.3 mo |

Univariate Kaplan Meier survival analysis of these patients based on clustered AQUA® scores revealed that these markers were indeed inversely related to disease-specific survival (phosphoGSK3B ser9 p-value<0.05).

Spearman-Rho analysis identified strong direct correlations between PhosphoCREB ser133 and PhosphoS6 ser240/244, and between PhosphoCREB ser133 and PhosphoGSK3Bser9 expression in this cohort of patients.

### MCA

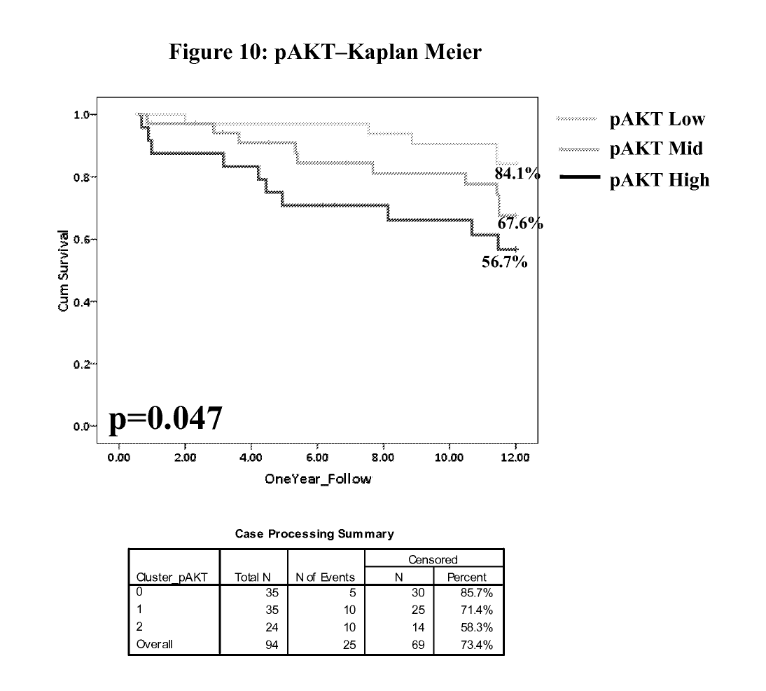

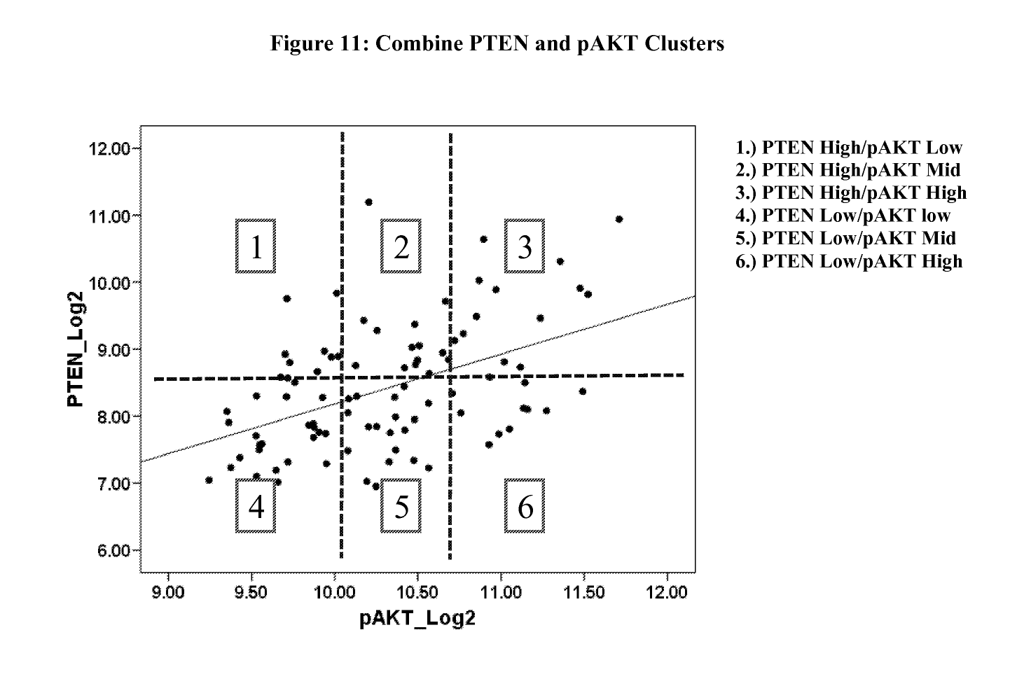

Multiparametric Correlative Discovery™ analysis is a method of multiple correspondence analysis that can provide insight into associations amongst biomarkers in a sampled population. In this study the MCA was constructed using cluster groups generated utilizing AQUA® scores. A biplot was generated to visualize associations (FIGS. 10, 11). This analysis indicated a strong association of the cluster of patients with low levels of phospho-protein expression and better survival.

### Summary

These data reveal that the signaling pathways targeted by Enzastaurin were activated specifically in patients with the poorest survival. These phosphomarkers, alone or in concert, are therefore useful for patient stratification and identification of patients best suited for Enzastaurin treatment

### PTEN/pAKT/mTOR Examples

### Cohort information

The HistoRx YTMA85 brain cancer cohort contains 110 GBM patient samples at 2× redundancy with a median follow-up time of 13.2

### Staining Protocol

Paraffin sections were deparaffinized in xylene and hydrated and then put in Tris EDTA buffer PT Module™ Buffer 4 (100× Tris EDTA Buffer, pH 9.0) TA-050-PM4X (Lab Vision Corp, Fremont Calif.) for antigen retrieval. Sections were then rinsed once in 1×TBS Tween (Lab Vision, Fremont, Calif.) for 5 minutes and incubated in peroxidase block (Biocare Medical, Concord, Calif.) for 15 min followed by a rinse in 1×TBS Tween for 5 min. Sections were blocked using Background Sniper (Biocare Medical, Newport Beach, Calif.) for 15 min. Sections were incubated with the primary antibody cocktail: rabbit anti-biomarker antibody and mouse anti-GFAP (DAKO, lot #M076101-2 at a 1:100 concentration) diluted in DaVinci Green (Biocare Medical, Newport Beach, Calif.) for 1 hours at room temp. In this study rabbit anti-biomarker antibodies included: PTEN at a dilution of 1:25 (Cell Signaling Technology, clone 138G6, CAT#9559); mTOR as a dilution of 1:50 (Cell Signaling Technology, clone 7C10, CAT#2983); pmTOR at a dilution of 1:10 (Cell Signaling Technology, clone 49F9, CAT#2976); and pAKT at a dilution of 1:25 (Cell Signaling Technology Clone 736E11, CAT#3787). Following three 5 min. rinses in 1×TBS Tween, slides were incubated in secondary antibody cocktail of goat anti-rabbit EnVision (DAKO, prepared per manufacturer's instructions) and goat anti-mouse Alexa Fluor 555 (Invitrogen A21429 diluted 1:200 into the EnVision) for 30 minutes in the dark, rinsed and then treated with Cy5 tyramide, diluted 1:50 in amplification buffer (Perkin Elmer SAT705A) for 10 min. room temperature in the dark, mounted with Prolong anti-fade with DAPI (Invitrogen, Carlsbad Calif.) and allowed to dry overnight.

Each stained specimen was imaged using a PM-2000™ system (HistoRx, New Haven Conn.) at 20× magnification. A board-certified pathologist reviewed an H&E stained serial section of the glioblastoma cohort to confirm tumor tissue presence in the samples. Images were evaluated for quality prior to analysis as described in co-pending U.S. Application 60/954,303. AQUA® analysis of the biomarkers was conducted and the biomarkers are quantified within cytoplasmic and nuclear compartments as described in Camp et al 2002 Nature Medicine 8(11)1323-1327.

### Results

AQUA® score distribution frequency analysis and histograms were generated for biomarker expression in the tissue samples of the GBM cohort. PTEN expression AQUA® scores obtained from analysis of the GBM cohort ranged from 123 to 2344 with a median of 314. mTOR expression AQUA® scores ranged from 112 to 1377, with a median of 405 (FIG. 1). Expression of PTEN and mTOR by AQUA analysis in 110 cases of GBM found no quantitative correlation between the two biomarkers (R=0.125; p=0.23).

### PTEN

Two-step unsupervised cluster analysis of PTEN AQUA® scores from the GBM cohort showing patients could be segregated into two groups, one with low PTEN expression (49% of patients) and a second with high PTEN expression (39% of patients) (FIG. 2).

Kaplan-Meier survival analysis shows a significant (p=0.043) 25.5% reduction in three-year disease specific survival between patients with PTEN-high and PTEN-low expressing tumors. Patients with high PTEN expression showed an 8.4 month improved median three-year disease specific survival rate from 15.6 months to 24.0 months (19.7% to 43.2% survival) and this was significant at the 10% level (p=0.062) (FIG. 3).

Univariate Cox proportional hazards analysis on both categorical (clusters) and continuous AQUA® data showing a significant HR=0.564 (95CI: 0.32-0.99; p=0.048) for PTEN cluster groupings and a significant HR=0.727 (95CI: 0.54-0.98; p=0.034) for AQUA® scores taken on a continuous basis. These data confirm the Kaplan-Meier survival analysis but also suggest that PTEN AQUA® scores could be used in a continuous rather than categorical fashion to predict survival

mTOR

Two-step unsupervised cluster analysis of mTOR AQUA® scores from the GBM cohort showing patients could be segregated into two groups, one with low mTOR expression (39% OF PATENTS) and a second with high mTOR expression (49% of patients) (FIG. 4). Kaplan-Meier survival analysis shows a non-significant (p=0.206) 19.7% reduction from 38.0 to 18.3% in three-year disease specific survival between patients with mTOR-high and mTOR-low expressing tumors. Patients with high mTOR expression showed a 6.1 month improved median three-year disease specific survival rate from 16.2 to 22.3 (FIG. 5). There was not a significant association between continuous mTOR AQUA scores and survival.

Univariate Cox proportional hazards analysis on both categorical (clusters) and continuous AQUA® data showing a non-significant HR=0.706 (95CI: 0.41-1.22; p=0.212) for mTOR cluster groupings and a non-significant HR=0.796 (95CI: 0.53-1.19; p=0.266). These data confirm the Kaplan-Meier survival analysis and suggest that mTOR AQUA® scores should not be used on a continuous basis to predict survival in GBM.

Multiplexed PTEN, mTOR Results:

Taking advantage of the continuous nature of the AQUA® scores for PTEN and mTOR, AQUA® data can be multiplexed to produce a novel combined biomarker assay. Plotting PTEN AQUA® scores versus mTOR AQUA® scores and using the unsupervised clustering cutpoints, four groups representing low/low, high/low, low/high, and high/high PTEN/mTOR expression respectively were created (FIG. 6). The median disease free survival for the high/high group exceeded 36 months (53.5% disease specific survival at 36 months). This association was significant at the 10% level (p=0.071). Comparing the high/high to all others showed a significant 31.9% increase from 21.6 to 53.5% in three-year disease specific survival (p=0.011). Median survival for this group was 15.7 months.

Univariate Cox proportional hazards analysis on groupings as defined in FIG. 6 demonstrate a significant HR=0.419 (95CI: 0.21-0.84; p=0.014). These data confirm the Kaplan-Meier survival analysis.

pmTOR

The pmTOR expression AQUA® scores ranged from 195 to 4869, with a median of 710 (FIG. 8). Expression of pmTOR by AQUA analysis in 110 cases of GBM found a positive linear quantitative correlation between pmTOR and mTOR (R=0.348; p=0.001) and pAKT (R=0.544; p<0.001) but not PTEN (R=0.188; p=0.08).

Two-step unsupervised cluster analysis of pmTOR AQUA® scores from the GBM cohort showing patients could be segregated into two groups, one with low pmTOR expression (65.2% of patients) and a second with high pmTOR expression (34.8% of patients). Kaplan-Meier survival analysis showed no association of pmTOR expression and disease specific survival.

pAKT

The pAKT expression AQUA® scores obtained from analysis of the GBM cohort ranged from 606 to 3351 with a median of 1252. (FIG. 8) Expression of pAKT by AQUA analysis in 110 cases of GBM found a positive linear quantitative correlation between the PTEN (R=0.470; p<0.001), mTOR (R=0.374; p<0.001), and pmTOR (R=0.544; p<0.001).

Two-step unsupervised cluster analysis of pAKT AQUA® scores from the GBM cohort showing patients could be segregated into three groups, one with low pAKT expression (25.5% of patients); a mid pAKT expressing group (31.2% of the patients; and a high pAKT expressing group (37.2% of patients) (FIG. 9).

Kaplan-Meier survival analysis shows a significant 27.4% decrease in one-year disease-specific survival from 84.1% to 56.7% for pAKT-low versus pAKT-high (FIG. 10) However at three years pAKT expression was not statistically significantly associated with survival prediction.

Multiplexed PTEN, pAKT Results:

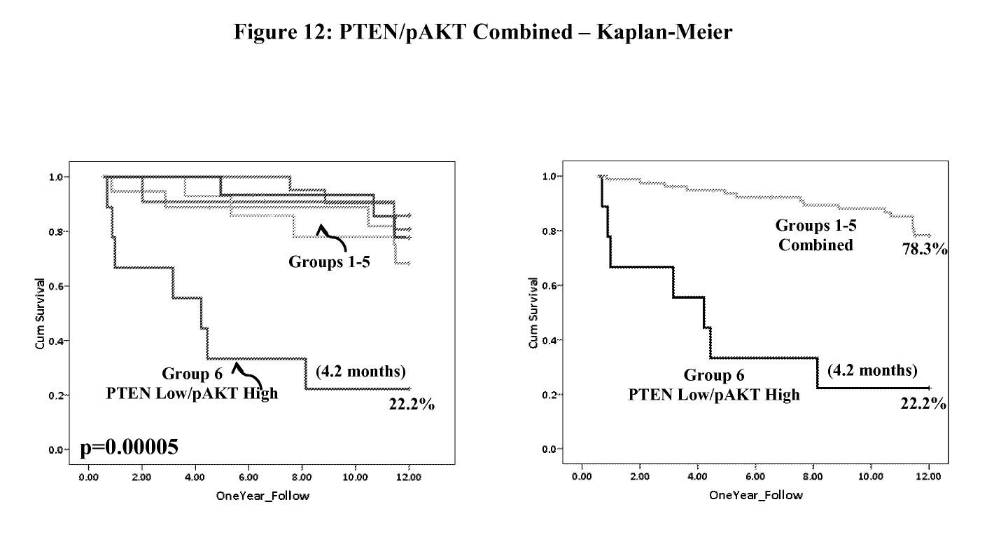

Taking advantage of the continuous nature of the AQUA® scores for PTEN and pAKT, AQUA® data can be multiplexed to produce a novel combined biomarker assay. Plotting PTEN AQUA® scores versus pAKT AQUA® scores and using the unsupervised clustering cutpoints, six groups representing low/low, low/mid, low/high, high/low, high/mid and high/high PTEN/pAKT expression respectively were created (FIG. 11). The median disease free survival for the low/high group was only 4.2 months (22.2% disease specific survival at 12 months). This association was highly significant at one year (p=0.00005) and three years (p=0.004). As depicted in FIG. 12 (right), the comparison of the low/high group to all others showed a significant 56.1% decrease from 78.3% to 22.2% in 1-year disease specific survival (p=0.00000007).

### Cox Model(s)

### One Year

In order to take broad advantage of data from the pathway markers studied and to develop a robust clinical model that can be used to broadly ascertain a patient's risk, a Cox proportional hazards model was derived for predicting survival at one year based on continuous expression data for each of the markers. Two models were developed:

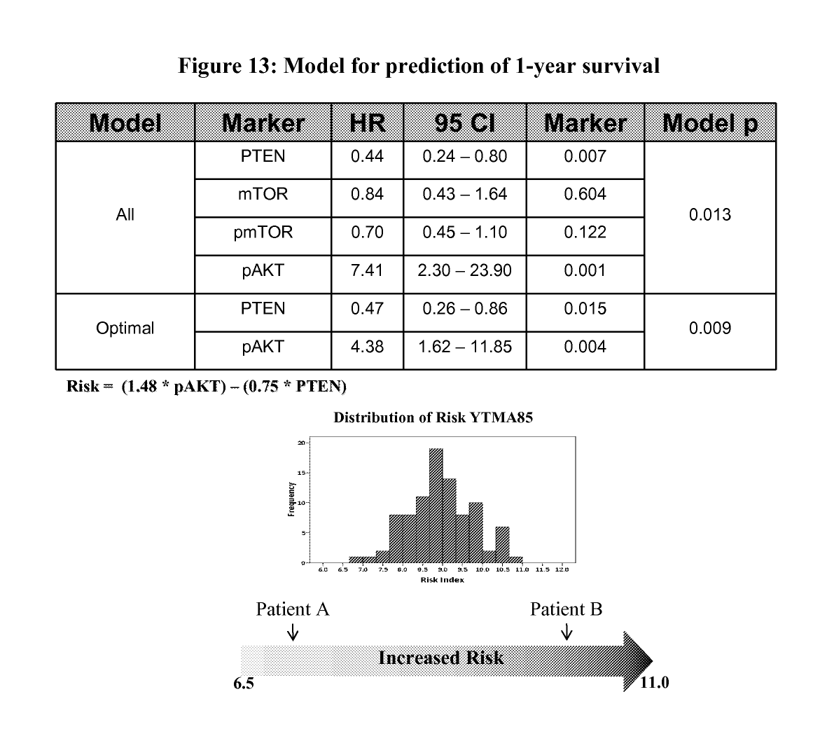

1.) Keeping all markers in model resulted in a significant model (p=0.013) with PTEN and pAKT contributing significantly to the model (p=0.007 and p=0.001 respectively) and mTOR and pmTOR not contributing significantly (FIG. 13):

Risk=(1.9*pAKT)−(0.785*PTEN)−(0.177*mTOR)−(0.353*pmTOR) Model 1:

2.) Optimization created a highly significant model (p=0.009) with only PTEN and pAKT in the model, both contributing significantly (p=0.015 and p=0.004):

Risk=(1.5*pAKT)−(0.75*PTEN) Model 2:

### Three Year

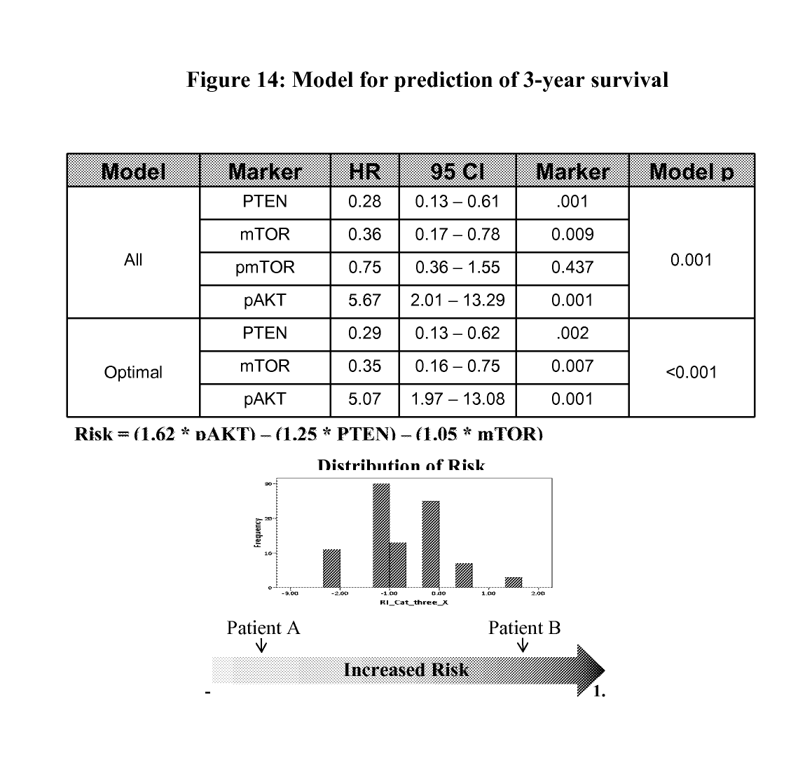

For prediction of three-year disease specific survival, a Cox proportional hazards model was derived for predicting survival at three years based on categorical expression data for each markers. Expression scores are put into low and high categories based on their univariate optimal cutpoint as determined by X-tile (FIG. 14). Two models were developed:

1.) Keeping all markers in model resulted in a highly significant model (p=0.001) with PTEN, mTOR, and pAKT contributing significantly to the model (p=0.001, p=0.009, and p=0.001 respectively) and pmTOR not contributing significantly (FIG. 14):

Risk=(1.6*pAKT)−(1.27*PTEN)−(1.01*mTOR)−(0.29*pmTOR) Model 1:

2.) Creating an optimal model resulted in a highly significant model (p=0.0004) with only PTEN, mTOR, and pAKT in the model, both contributing (p=0.002, p=0.007, and p=0.001 respectively) significantly:

Risk=(1.6*pAKT)−(1.25*PTEN)−(1.05*mTOR) Model 2:

From all of these models, a risk continuum can be generated whereby a individual patients, based on their expression levels of these biomarkers, can be placed on this continuum and clinical decisions made thereof (see FIGS. 13 and 14).

Tissue Microarrays (TMA) containing 110 primary glioblastomas at two fold redundancy were formalin fixed, paraffin-embedded tumor samples obtained at Yale University-New Haven Hospital from 1961-1983 and was constructed at the Yale University Tissue Microarray Facility. The median follow-up time is 13.2 months.

Immunohistochemistry (IHC). A modified indirect immunofluorescence protocol, with heat-induced epitope retrieval in Tris-EDTA buffer (pH 9.0) as described previously (Camp et al. Automated subcellular localization and quantification of protein expression in tissue microarrays. 2002 Nature Medicine. 11:1323) All antibodies were from Cell Signaling Technology (Danvers, Mass.). Staining conditions for PTEN antibody (Clone 138G6 rabbit monoclona) at 1:25), mTOR antibody (Clone 7C10 rabbit monoclonal), pmTOR antibody (Clone 49F9 mouse monoclonal), and pAKT (Clone 736E11 rabbit monoclonal) were quantitatively optimized using test-arrays containing a sampling of glioblastoma tissue cores. Dilutions of 1:25, 1:50, 1:10, and 1:25 respectively were determined to be optimal.

AQUA Analysis Specific expression as measured by indirect fluorescent antibody binding was determined by normalized pixel intensity within specific tumor compartments as described previously (Camp et al. and FIG. 1) and through HistoRx's developed algorithms.

Statistics. Expression, regression and survival analysis was performed using SPSS™ (Version 14.0). Hierarchical clustering (average linkage analysis) was performed using Cluster from Micheal Eisen's Laboratory <URL: http://rana.stanford.edu/sofrtware>.

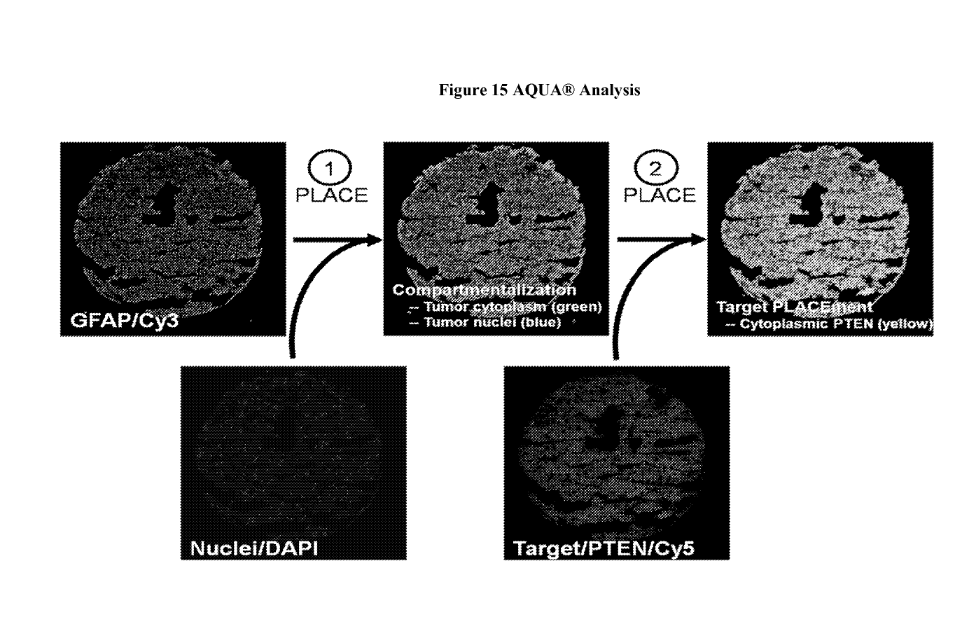

FIG. 15: AQUA® Analysis. Taking advantage of the multiplexing power of fluorescence staining, cellular compartments and/or target genes can be labeled differentially. Tumor-specific cytoplasm is labeled with GFAP (neuronal-specific) in the Cy3 channel, while nuclei are labeled with DAPI in the UV channel. (1) Using Pixel-based locale assignment for compartmentalization (PLACE) algorithms, pixels can be designated as either nucleus or cytoplasm. (2) Using PLACE again, target pixels (i.e. PTEN used here) can be assigned to specific compartments. Target pixel intensities are then summed and normalized for compartment size and exposure time to produce an AQUA® score.

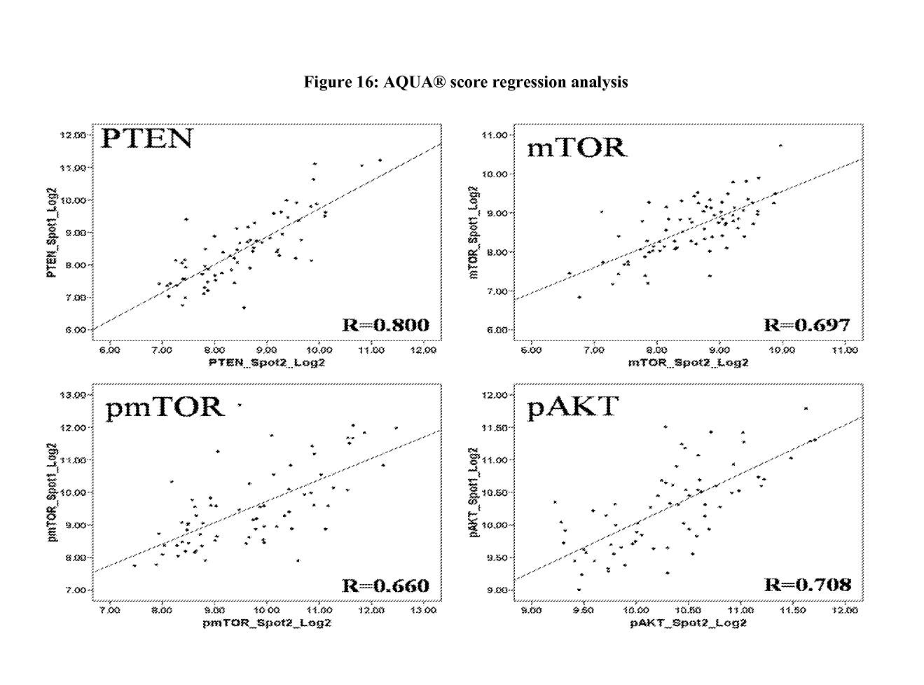

FIG. 16: AQUA® score regression analysis. Given for each indicated biomarker are scatterplots and Pearson R-values for AQUA® scores (log² transformed) between redundant tissue cores from YTMA85. AQUA® analysis demonstrates significant reproducibility for each biomarker tested.

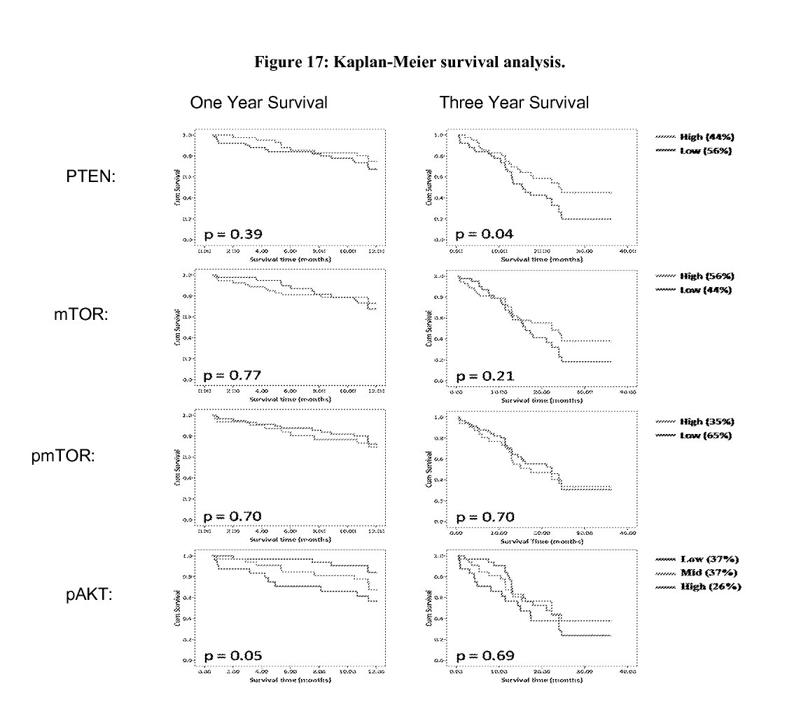

FIG. 17: Kaplan-Meier survival analysis. Unsupervised clustering analysis was performed for each indicated biomarker to segment the patient population based on AQUA® scores. Populations for each biomarker were divided as indicated at right. One-year (left column) and three-year (right column) disease-disease specific Kaplan-Meier survival analysis was performed with indicated log-rank p-values. PTEN expression did not predict one-year survival, but high PTEN expression significantly associated with improved three-year survival [8.3 month increase in median survival from 15.6 (PTEN low) to 24.0 months (PTEN high); p=0.043]. Although mTOR expression did not significantly predict one-year or three-year survival, there was a trend toward improved three-year survival [5.5 month increase in median survival from 16.2 (mTOR low) to 22.3 months (mTOR high); p=0.021]. pmTOR did not predict one-year or three year survival. Elevated pAKT expression significantly associated with decrease overall survival [27.4% decrease in cumulative survival from 84.1 to 56.7%; p=0.05].

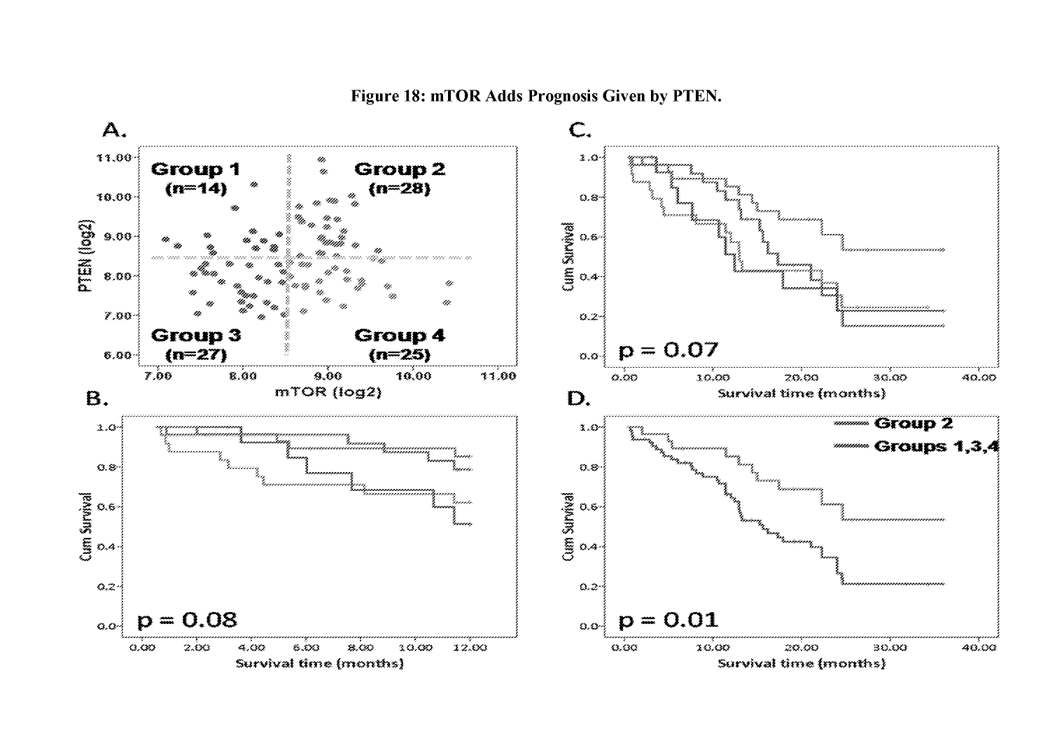

FIG. 18: mTOR adds prognosis given by PTEN. A.) Scatterplot between PTEN and mTOR AQUA® showing divisions and color coding based on cutpoints from FIG. 3 [Group 1: PTEN high/mTOR low; Group 2: PTEN high/mTOR high; Group 3: PTEN low/mTOR low; Group 4: PTEN low/mTOR high]. B.) One-year disease specific Kaplan-Meier Survival analysis showing an association between Groups 2 and 3 (both high or both low) with improved survival (p=0.08). C.) Three-year disease specific Kaplan-Meier survival analysis showing an association between Group 2 (PTEN high/mTOR high) and improved survival (p=0.07). D.) As suggested in 4C, Groups 1,3, and 4 were combined and survival compared to Group 2 (PTEN high/mTOR high) giving a significant (p=0.01) association with survival showing at least a 19.3 month improvement in median survival from 15.7 (combined groups) to >36 months for patients with high PTEN and high mTOR.

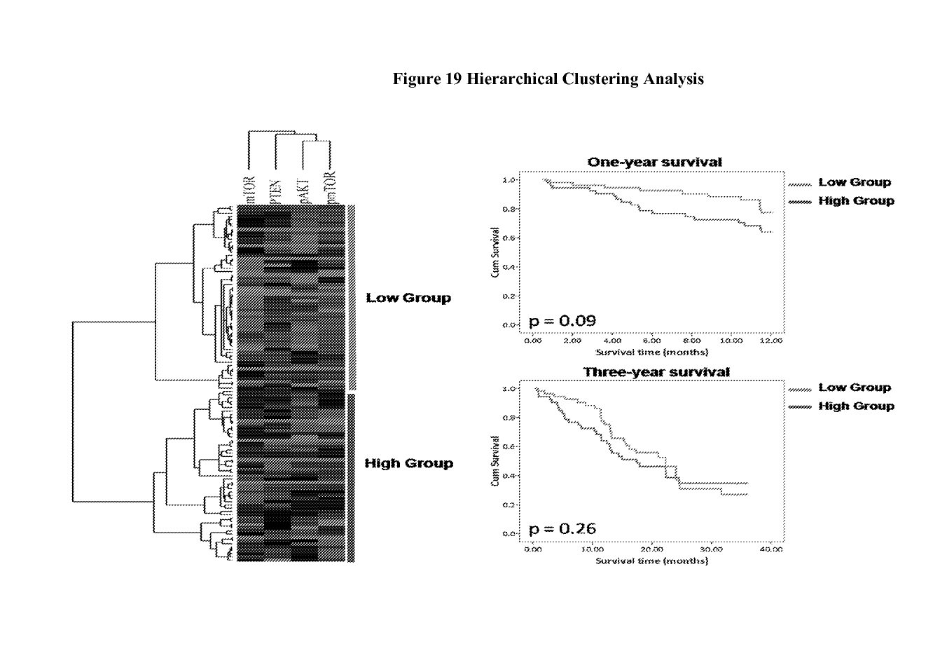

FIG. 19: Hierarchical clustering analysis. Hierarchical clustering was performed (heat map) and demonstrated two predominant populations of patients with respect to the four biomarkers analyzed: a low group and a high group (see heat map). One-year survival disease-specific Kaplan-Meier survival analysis revealed an association between the high group and decreased overall survival (13.6% decrease from 77.6 to 64%; p=0.09). These groups were not predictive at three years.

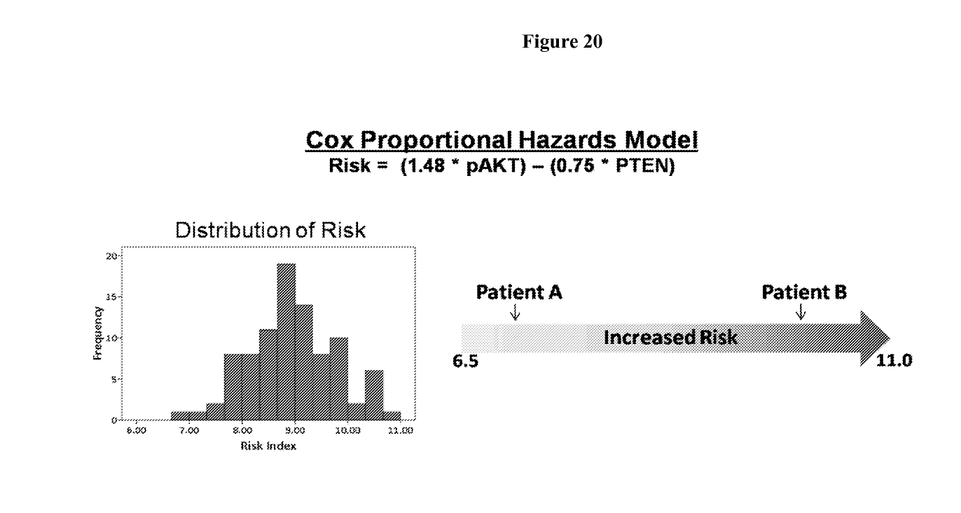

FIG. 20: Cox Proportional Hazards Model

TABLE I
Marker Correlation.

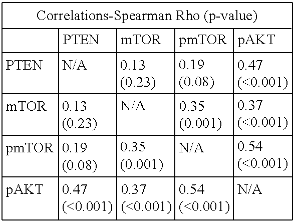

All four markers were analyzed to determine whether quantitative correlations exist. Rank-order analysis was performed with given Spearman's Rho and p-values. Boxes in green indicate significant correlations.

TABLE II
Cox proportional hazards model using continuous
AQUA ® scores.

| Model | Marker | HR | 95 CI | Marker p | Model p |
|---|---|---|---|---|---|
| All | PTEN | 0.44 | 0.24-0.80 | 0.007 | 0.013 |
| | mTOR | 0.84 | 0.43-1.64 | 0.604 | |
| | pmTOR | 0.70 | 0.45-1.10 | 0.122 | |
| | pAKT | 7.41 | 2.30-23.90 | 0.001 | |
| Optimal | PTEN | 0.47 | 0.26-0.86 | 0.015 | 0.009 |
| | pAKT | 4.38 | 1.62-11.85 | 0.004 | |

Two models with indicated hazard ratios (HR), 95% confidence intervals (95 CI), p-values for marker (Marker p), and p-values for the model (Model p). The first model (All; p = 0.013) keeps in all markers, but not all markers contribute significantly. The second model (Optimal; p = 0.009) keeps only markers in the model that contribute significantly.

### SUMMARY AND CONCLUSIONS

AQUA® analysis of PTEN, mTOR, pmTOR, and pAKT in glioblastoma is highly reproducible.
By AQUA® analysis: 1.) high PTEN expression predicts improved 3-year survival; 2.) high pAKT expression predicts decreased 1-year survival.
Combining mTOR with PTEN predicts for a group of patients that do relatively well and better than as predicted by PTEN and/or mTOR alone.
In this cohort, a signficiant multivariate Cox proportional hazards model using continuous AQUA® scores was generated that can predict the relative one-year survival risk for a patient.

What is claimed is:

1. A method of determining a prognosis of a patient suffering from a medical condition comprising: determining the expression level of at least one protein biomarker, and/or a phosphorylated form thereof, associated with a PI3K/AKT/mTOR pathway in a tissue specimen obtained from the patient, and assessing the patient's prognosis from the determined expression level.

2. The method of claim 1, wherein said at least one biomarker is selected from one or more of the group consisting of: GSK3β, S6, CREB, PTEN, AKT, mTOR and/or phosphorylated forms thereof.

3. The method of claim 1, in which said medical condition includes brain cancer.

4. The method of claim 3, in which said brain cancer is characterized as glioblastoma multiforme.

5. The method of claim 1, wherein said at least one biomarker is selected from one or more of the group consisting of GSK3β, S6 and CREB, wherein a low expression level is indicative of a relatively good prognosis and a high expression level is indicative of a relatively poor prognosis.

6. The method of claim 1, wherein said at least one biomarker is PTEN, wherein a high expression level is indicative of a relatively good prognosis and a low expression level is indicative of a relatively poor prognosis.

7. The method of claim 6, wherein the expression level of a second biomarker, is determined, in which a high expression level of PTEN and a high expression level of mTOR are indicative of a relatively good prognosis, and in which any other resulting combination of expression levels (i.e., high PTEN/low mTOR, low PTEN/low mTOR, or low PTEN/high mTOR) is indicative of a relatively poor prognosis.

8. The method of claim 6, wherein the expression level of a second biomarker, pAKT, is determined, in which a low expression level of PTEN and a high expression level of pAKT are indicative of a relatively very poor prognosis, and in which low PTEN/low pAKT, low PTEN/medium pAKT, high PTEN/low pAKT, high PTEN/medium pAKT or high PTEN/high pAKT is indicative of a relative good prognosis.

9. The method of claim 1, wherein said patient is naïve or is undergoing treatment with an inhibitor of the P13 kinase/AKT/mTOR pathway.

10. The method of claim 9, wherein said inhibitor is selected from the group consisting of Enzastaurin and rapamycin, or pharmaceutically acceptable salts thereof and optionally combined with temozolomide, radiation, or both.

11. A method of assessing a prognosis of a patient suffering from a medical condition comprising:
a) providing, obtaining or receiving a tissue sample from a patient suffering from a medical condition;
b) incubating the tissue sample with a first stain that specifically labels a first marker-defined subcellular compartment, a second stain that specifically labels a second marker-defined subcellular compartment, and one or more additional stains, each additional stain labeling a specific biomarker selected from the group consisting of: GSK3β, S6, CREB, PTEN, AKT and mTOR, and/or phosphorylated forms thereof.
c) obtaining an image of each of the first, the second and the one or more additional stains;
d) deriving from at least some of said images a staining score indicative of an expression level of each specific biomarker in the first compartment or the second compartment or both; and
e) assessing from the resulting expression levels the patient's prognosis.

12. The method of claim 11, in which said medical condition includes brain cancer.

13. The method of claim 11, in which said brain cancer is characterized as glioblastoma multiforme.

14. The method of claim 11, which includes one additional stain for specifically labeling PTEN and another additional stain for specifically labeling mTOR and in which a finding of high expression level of PTEN together with a high expression level of mTOR is indicative of relatively good prognosis.

15. The method of claim 11, which includes a first stain for specifically labeling a marker, which defines cytoplasm.

16. The method of claim 12, which includes a first stain specifically labeling a marker, said marker including GFAP.

17. A kit comprising:
a) one or more stains, each labeling a specific biomarker selected from the group consisting of: GSK3β, phosphorylated GSK2β, S6, phosphorylated S6, CREB, phosphorylated CREB, PTEN, AKT, phosphorylated pAKT, mTOR, phosphorylated mTOR, and/or phosphorylated forms thereof
b) optionally, a first stain specific for a first subcellular compartment of a cell;
c) optionally, a second stain specific for a second subcellular compartment of the cell; and
d) instructions for using the kit.

18. The kit of claim 17, in which said optional first stain is specific for a cytosolic compartment of the cell.

19. The kit of claim 17, in which said optional second stain is specific for a nuclear compartment of the cell.

20. The kit of claim 17, in which said optional second stain includes DAPI.

21. A method of identifying a patient suitable for treatment with a pharmaceutical inhibitor of a PI3K/AKT/mTOR pathway comprising: determining an expression level of at least one protein biomarker associated with a PI3K/AKT/mTOR pathway in a tissue specimen obtained from the patient,
wherein the expression level of said at least one biomarker is indicative of whether the patient is more likely or less likely to benefit from a treatment with a pharmaceutical inhibitor of said PI3K/AKT/mTOR pathway.

22. The method of claim 21, wherein the predicted benefit of a specific treatment ranges from a one to a three-year period.

23. The method of claim 21, wherein the predictive benefit of a specific treatment is evaluated from consideration of expression levels of one or more of at least seven specific protein biomarkers from selected from the group consisting of GSK3β, S6, CREB, PTEN, AKT, mTOR, and/or phosphorylated forms thereof.

24. The method of claim 1, wherein said expression level is characterized as low, medium or high.

25. The method of claim 24, wherein a low to intermediate protein concentration level of nuclear GSK3β represent a range of AQUA® scores from about 300 to about 2000.

26. The method of claim 24, wherein a high protein concentration level of nuclear GSK3β represent a range of AQUA® scores from about 2000 to about 4000.

27. The method of claim 24, wherein a low to intermediate protein concentration level of cytoplasmic phosphorylated GSK3β represent a range of AQUA® scores from about 500 to about 1500.

28. The method of claim 24, wherein a high protein concentration level of cytoplasmic GSK3β represent a range of AQUA® scores from about 1500 to about 2500.

29. The method of claim 24, wherein a low to intermediate protein concentration level of nuclear phosphorylated CREB represent a range of AQUA® scores from about 250 to about 3000.

30. The method of claim 24, wherein a high protein concentration level of nuclear phosphorylated CREB represent a range of AQUA® scores from about 3000 to about 6000.

31. The method of claim 24, wherein a low protein concentration level of PTEN represent a range of AQUA® scores from about 200 to about 260.

32. The method of claim 27, wherein a high protein concentration level of PTEN represent a range of AQUA® scores from about 300 to about 800.

33. The method of claim 27, wherein a low protein concentration level of mTOR represent a range of AQUA® scores from about 200 to about 300.

34. The method of claim 27, wherein a high protein concentration level of mTOR represent a range of AQUA® scores from about 300 to about 800.

35. The method of claim 27, wherein a low protein concentration level of phosphorylated AKT represent a range of AQUA® scores from about 800 to about 1024.

36. The method of claim 27, wherein an intermediate protein concentration level of phosphorylated AKT represent a range of AQUA® scores from about 1024 to about 1500.

37. The method of claim 27, wherein a high protein concentration level of phosphorylated AKT represent a range of AQUA® scores from about 1500 to about 3000.
